(19) United States
(12) Patent Application Publication
(10) Pub. No.: US 2026/0263584 A1
(43) Pub. Date: Sep. 10, 2026

(54) Modulating the Innate Immunity of RNA

(71) Applicant: Pfizer Inc., New York, NY (US)

(72) Inventors: Pirada Suphaphiphat Allen, Congers, NY (US); Justin Leigh Bellenger, Cheshire, CT (US); Ye Che, Niantic, CT (US); Liyang Cui, Woodcliff Lake, NJ (US); Ivna Padron De Souza, Nyack, NY (US); Regis Doyonnas, Stonington, CT (US); Qianjing He, Nanuet, NY (US); Sue-Jean Hong, Montvale, NJ (US); Karina Ng Huang, Brooklyn, NY (US); Thomas Schroeter, Preston, CT (US); Shuai Shi, Whippany, NJ (US); Matthew Drew Troutman, Mystic, CT (US); Lawrence Gabriel Webb, New Rochelle, NY (US); Cindy Xudong Yang, Tappan, NY (US); Herg Hwa Zhang, Pearl River, NY (US)

(21) Appl. No.: 19/165,220

(22) PCT Filed: Mar. 14, 2024
(86) PCT No.: PCT/IB2024/052474

§ 371 Date: Sep. 15, 2025

Related U.S. Application Data
(60) Provisional application No. 63/490,787, filed on Mar. 17, 2023.
(60) Provisional application No. 63/556,506, filed on Feb. 22, 2024.
(60) Provisional application No. 63/556,623, filed on Feb. 22, 2024.

(51) Int. Cl. A61K 39/145; A61K 9/1272; A61K 9/51; A61K 31/4725; A61K 31/517; A61K 39/00; C12N 7/00; C12N 15/67; C12N 15/88

(52) U.S. Cl. CPC A61K 39/145; A61K 9/1272; A61K 9/5123; A61K 31/4725; A61K 31/517; C12N 7/00; C12N 15/67; C12N 15/88; A61K 2039/53; A61K 2039/55555; A61K 2039/70; C12N 2760/16134

(57) ABSTRACT

The present disclosure provides methods and compositions for modulating the innate immunity to exogenous ribonucleic acid (RNA) when administered to a subject in need thereof. More particularly, the disclosure provides compositions comprising RNA encoding at least one antigen or therapeutic protein, and at least one compound that modulates the innate immunity to such RNA when administered to a subject in need thereof. The disclosure further provides methods of using such compounds to modulate the innate immunity of such compositions.

### FIELD

The present disclosure provides methods and compositions for modulating the innate immunity of exogenous ribonucleic acid (RNA) when administered to a subject in need thereof. More particularly, the disclosure provides compositions comprising RNA encoding at least one antigen or therapeutic protein, and at least one compound that modulates the innate immunity of such RNA when administered to a subject in need thereof. The disclosure further provides methods of using such compounds to modulate the innate immunity of such RNA.

### BACKGROUND

Over the last 30 years, RNA has been shown to be capable of producing therapeutic and prophylactic benefits in cells and complex organisms, and more recently, RNA-based drugs have been approved for use in subjects. RNA's potential as a therapeutic and prophylactic agent is an active area of biomedical research, and this area is expected to mature rapidly in the coming years. To date, RNA has been used to target nucleic acids and proteins, and to encode proteins. Single-strand antisense oligonucleotides and double-strand interfering RNA's can prevent protein synthesis by preventing transcription and/or translation. RNA can also alter protein function directly by forming aptamers to control protein activity, thereby realizing a therapeutic goal related to altering protein function. Finally, messenger RNAs (mRNAs) can be used to produce desired proteins, such as antigens in the context of vaccination. For example, important biomedical uses of mRNAs include vaccines and protein replacement therapy (Sarah Deweerdt, Nature, Vol. 574, 17 Oct. 2019). Self-amplifying RNA (saRNA) has received significant attention as a potential platform for vaccination due to its capacity to encode multiple antigens because of its larger size, and its ability to self-replicate. This ability to self-replicate leads to enhanced and prolonged antigen expression, which helps overcome the transient nature of antigen expression in non-amplifying mRNA vaccines (Luis A. Brito et al., Advances in Genetics Vol 89, pp. 179-233 (2015). saRNA is also beneficial since it can typically be delivered at lower concentrations than non-amplifying mRNA and still achieve similar benefit (A. B. Vogel et al., Mol. Ther. 26(2), pp. 446-455 (2018)).

While it is clear that RNA can have beneficial therapeutic and prophylactic effects, a major challenge to its use involves the innate immune response that occurs when exogenous RNA is administered to a subject. Because of its larger size and significant secondary structure, saRNA in particular can often trigger a significant innate immune response that leads to interferon secretion, which can decrease RNA translation and promote RNA degradation (J. Devoldere et al., Drug Discover. Today 21(1), pp. 11-25 (2016); A. K. Blakney et al., Vaccines Spec. Issue 9, pp. 1-26 (2021)). For mRNA applications not involving immunotherapy, this innate immune response is completely undesirable. For applications that involve immunotherapy (such as vaccines), however, the goal is typically to modulate the innate immune response rather than eliminate it altogether. Strategies being investigated to modulate the innate immune response include: modifications to the mRNA backbone (e.g. 5′ cap structure, poly-A tail, nucleoside bases, open reading frames (i.e. via codon optimization), and sequences affecting secondary structure); optimization of the in vitro transcription and subsequent purification of the mRNA; and the use of innate immune inhibitors (A-K Minnaert et al., Adv Drug Deliv. Rev. 176 (2021)).

Significantly, nucleoside base modification (one of the key methods of modulating the innate immune response to mRNA) is not considered possible in the case of saRNA since such modified nucleosides would be lost after the first round of amplification within the cell. Further, such modified nucleobases are believed to reduce the ability of saRNA to self-replicate in the target cells (G. Maruggi et al., Mol. Ther. 27(4), pp. 757-772 (2019)). Accordingly, there is a need in the art for methods of effectively modulating the innate immune response to RNA therapeutic modalities, and in particular to saRNA. The present disclosure provides novel methods of using small molecule compounds in combination with RNA therapies in order to modulate the innate immune response to the RNA. The present disclosure further provides novel compositions that comprise such small molecule compounds in combination with RNA therapies.

### SUMMARY

The present disclosure provides methods of modulating the innate immune response when exogenous RNA is administered to a subject. For example, the present disclosure provides methods of using particular small molecule compounds in combination with the administration of RNA (e.g. saRNA in the context of vaccination) to effectively modulate the innate immune response to such RNA. For example, such small molecules can be used in combination with saRNA that encodes at least one antigen from influenza (flu) virus.

The present disclosure further provides compositions comprising at least one such small molecule compound in combination with at least one RNA construct (e.g. saRNA).

The small molecules that can be used within the scope of the compositions and methods disclosed herein include modulators (such as inhibitors and/or degraders) of interleukin-1 receptor associated kinase 4 (IRAK4) and of retinoic acid-inducible gene I (RIG-1).

### BRIEF DESCRIPTION OF THE FIGURES

FIG. 1 provides A/Wisc/588/19 HA expression in Hela cells with or without the IRAK4 degrader (PF-07911620). Hela cells were transfected with LNP containing saRNA-TC83-A/Wisc/588/19 HA-40A (RMM59, 100 ng) with (7.5 mM) and without IRAK4 degrader (PF-07911620). Number of live cells (% live cells) (A), number of HA expressing cells (% HA positive cells) (B), total HA expression per HA positive cell (Geometric mean fluorescence intensity (GMFI)) (C) were determined by flow cytometry at 20 to 24 hours post transfection, the relative transcript levels of subgenomic (left) and genomic saRNA (right) (D), were determined by RT-qPCR at 2 and 24 hours post transfection. (−) denotes vehicle control, DMSO. Results are presented as mean±standard deviation for each group from a representative experiment. *p<0.05 as evaluated using one-way ANOVA test with multiple comparisons.

FIG. 2 provides A/Wisc/588/19 HA expression in Hela cells with or without the IRAK4 degrader (PF-07932784). Hela cells were transfected with LNP containing saRNA-TC83-A/Wisc/588/19 HA-40A (RMM59, 125 ng) with (15, 7.5, 3.75, and 1.8 mM) and without IRAK4 degrader (PF-07932784). Number of live cells (% live cells) (A), number of HA expressing cells (% HA positive cells) (B), total HA expression per HA positive cell (Geometric mean fluorescence intensity (GMFI)) (C) were determined by flow cytometry at 20 to 24 hours post transfection, the relative transcript levels of subgenomic (left) and genomic saRNA (right) (D), were determined by RT-qPCR at 2 and 24 hours post transfection. (−) denotes vehicle control, DMSO. Results are presented as mean±standard deviation for each group from a representative experiment.

FIG. 3 provides A/Wisc/588/19 HA expression in Hela cells with or without RIG-1 compounds (PF-01034118 & PF-01009329). Hela cells were transfected with LNP containing saRNA-TC83-A/Wisc/588/19 HA-40A (RMM59, 125 ng) with (15, 7.5, 3.75, and 1.8 mM of PF-01034118 and 1.8, 0.9, 0.45, and 0.22 mM of PF-01009329) and without RIG-1 compounds. Number of live cells (% live cells) (A), number of HA expressing cells (% HA positive cells) (B), total HA expression per live cell (Mean fluorescence intensity (MFI)) (C) were determined by flow cytometry at 20 to 24 hours post transfection, the relative transcript levels of subgenomic (left) and genomic saRNA (right) (D), were determined by RT-qPCR at 24 hours post transfection. Results are presented as mean±standard deviation for each group from a representative experiment.

FIG. 4. A/Wisc/588/19 HA expression in HeLa with or without DPQ compounds (PF-01034118 & PF-01009329). HeLa cells were transfected with LNP containing modRNA-A/Wisc/588/19 HA-80A (RMM71, 29 ng) or saRNA-TC83-A/Wisc/588/19 HA-40A with or without 50% m1ψ incorporation (RMM59, 125 ng) with (15, 7.5, 3.75, & 1.8 mM of PF-01034118 or 1.8, 0.9, 0.45, & 0.22 mM of PF-01009329) and without DPQ compounds. Number of live cells (% live cells) (A), number of HA expressing cells (% HA positive cells) (B), total HA expression per live cell (Mean fluorescence intensity (MFI)) (C) were determined by flow cytometry at 20-24 hours post transfection. DMSO control denotes saRNA-LNP co-treatment with DMSO vehicle control. Results are presented as mean±standard deviation for each group from a representative experiment.

FIG. 5. A/Wisc/588/19 HA expression in HeLa with or without small molecules (PF-06650833 & PF-01009329). HeLa cells were transfected with LNP containing bicistronic saRNA-TC83-A/Wisc/588/19 HA-NA-80A with or without 50% m1ψ±100% m5C incorporation (125 ng) with (15, 5.0, & 1.7 mM of PF-06650833 or 1.8, 0.6, & 0.2 mM of PF-01009329) and without small molecules. Number of live cells (% live cells) (A), number of HA expressing cells (% HA positive cells) (B), total HA expression per live cell (Mean fluorescence intensity (MFI)) (C) were determined by flow cytometry at 20-24 hrs post transfection, the relative transcript levels of subgenomic saRNA (D), were determined by RT-qPCR at 2 & 12 hours post transfection. DMSO control denotes saRNA-LNP co-treatment with DMSO vehicle control. Results are presented as mean±standard deviation for each group from a representative experiment.

FIG. 6. A/Wisc/588/19 HA expression in HeLa with or without small molecule (PF-01009329). HeLa cells were transfected with LNP containing bicistronic saRNA-TC83-A/Wisc/588/19 HA-NA-80A with or without 50% m1ψ incorporation (125 ng) with (1.8, 0.6, & 0.03 mM of PF-01009329) and without small molecule. Number of live cells (% live cells) (A), number of HA or NA expressing cells (% HA or NA positive cells) (B), total HA expression per HA or NA positive cell (Geometric mean fluorescence intensity (GMFI)) (C) were determined by flow cytometry at 20-24 hours post transfection. DMSO control denotes saRNA-LNP co-treatment with DMSO vehicle control. Results are presented as mean±standard deviation for each group from a representative experiment.

FIG. 7. Neutralizing antibodies elicited by immunization of NHPs with LNP-formulated saRNA encoding Influenza HA & NA with or without small molecules (PF-06650833 & PF-01009329) measured by 1-day MNT. Female and male Cyno macaques were immunized IM with 2.5 μg of LNP-formulated influenza bicistronic saRNA (encoding A/Wisconsin/588/2019 (H1N1) HA-NA) incorporating either unmodified or modified nucleotides (50% m1ψ, 50% m1ψ+100% m5C) and co-administered with PF-06650833 at a 3.125 μg dose or PF-01009329 at a 0.14 μg dose or saline on Days 0 and 28. Neutralizing antibody titers against A/Wisconsin/588/2019 were measured in serum by a 1-Day microneutralization assay on day 42 (2 weeks post boost vaccination). 50% neutralization titers are plotted, and the geometic mean titer (GMT) 42 is displayed at the bottom of each bar. Dotted line indicates the limit of detection for the assay.

FIG. 8. A/Wisc/588/19 HA expression in HeLa with or without small molecule (PF-01009329). HeLa cells were transfected with LNP containing bicistronic saRNA-TC83-A/Wisc/588/19 HA-NA-80A with 50% m1ψ incorporation (11-point, 2-fold dilution series starting from 500 ng) with (18.1, 13.4, 10.0, & 6.5 mM of PF-01009329) and without small molecule. Number of live cells (% live cells) (A), number of HA expressing cells (% HA positive cells) (B), total HA expression per HA positive cell (Geometric mean fluorescence intensity (GMFI)) (C) were determined by flow cytometry at 24 hours post transfection. DMSO control denotes saRNA-LNP co-treatment with DMSO vehicle control. Results are presented as mean±standard deviation for each group from a representative experiment.

FIG. 9. A/Wisc/588/19 HA expression in HeLa with or without small molecules (PF-06650833 & PF-01009329) HeLa cells were transfected with LNP containing modRNA-A/Wisc/588/19 HA-80A (RMM71, 26 ng) with (15, 5.0, & 1.7 mM of PF-06650833 or 1.8, 0.6, & 0.2 mM of PF-01009329) and without small molecules. Number of live cells (% live cells) (A), number of HA expressing cells (% HA positive cells) (B), total HA expression per live cell (Mean fluorescence intensity (MFI)) (C) were determined by flow cytometry at 20-24 hours post transfection, the relative transcript levels of HA-encoding modRNA were determined by RT-qPCR at 2 & 12 hours post transfection (D). DMSO control denotes modRNA-LNP co-treatment with DMSO vehicle control. Results are presented as mean±standard deviation for each group from a representative experiment.

FIG. 10. Co-administration of PF-01009329 increased intracellular saRNA. HeLa cells were exposed to PF-01009329 3 hours prior to modRNA-LNP addition (Pre-treatment), PF-01009329 admixed with modRNA-LNPs (Co-treatment), or exposed to modRNA-LNPs for 1 hour prior to PF-01009392 addition (Post-treatment). [PF-01009329]=0.2 mM. The relative transcript levels of HA-encoding modRNA were determined by RT-qPCR at 0.5, 2, and 4 hours post transfection (A). DMSO control denotes modRNA-LNP co-treatment with DMSO vehicle control. Results are presented as mean±standard deviation for each group from a representative experiment. Representative images of HeLa cells were transfected with LNP containing saRNA-TC83-A/Wisc/588/19 HA-80A (RMM59, 24 ng) with (1.8, 0.6, 0.2 mM of PF-01009329) and without small molecule. Cells were fixed after 4 hours, processed for smFISH to fluorescently label saRNA (yellow spots), and imaged by fluorescence microscopy. Nuclei (blue) was detected by DAPI (B).

### DETAILED DESCRIPTION

Embodiments of the present disclosure provide compositions and methods that include RNA, e.g. a self-amplifying RNA (saRNA) polynucleotide encoding an influenza virus antigen, or other antigens, in combination with compounds that modulate the innate immune response to the RNA. RNA vaccines, such as influenza virus RNA vaccines, as provided herein may be used to induce a balanced immune response, comprising both cellular and humoral immunity.

It is contemplated that any embodiment discussed in this specification may be implemented with respect to any method or composition of the disclosure, and vice versa. Furthermore, compositions of the disclosure may be used to achieve methods of the disclosure.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

Described below are a number of embodiments (E) of the present disclosure.

E1. According to a first aspect of the present disclosure, there is provided a composition comprising at least one nucleic acid and at least one compound that modulates the innate immune response to said nucleic acid when administered to a subject in need thereof.

E2. The composition of embodiment E1, wherein the at least one nucleic acid is RNA.

E3. The composition of embodiment E2, wherein the RNA is modRNA.

E4. The composition of embodiment E2 wherein the RNA is saRNA.

E5. The composition of embodiment E4, wherein the saRNA comprises at least one open reading frame that encodes a gene of interest.

E6. The composition of embodiment E5, wherein the gene of interest is derived from influenza virus hemagglutinin (HA).

E7. The composition of embodiment E6, wherein the saRNA further comprises a second reading frame that encodes a second gene of interest.

E8. The composition of embodiment E7, wherein said second gene of interest is derived from influenza virus neuraminidase (NA).

E9. The composition of any one of embodiments E1 to E8 wherein the nucleotides are naturally-occurring.

E10. The composition of any one of embodiments E4 to E8 wherein at least 10% of the total nucleotides in the saRNA have been replaced with modified or unnatural nucleotides.

E11. The composition of embodiment E10, wherein the modified or unnatural nucleotides are selected from the group consisting of pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

E12. The composition of any one of embodiments E1 to E11, further comprising a cationic lipid.

E13. The composition of any one of embodiments E1 to E12, wherein the nucleic acid and the compound are encapsulated in, bound to, or adsorbed on a cationic lipid.

E14. The composition of any one of embodiments E1 to E13, further comprising a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, a polycationic peptide, or a cationic nanoemulsion.

E15. The composition of any one of embodiments E1 to E14, wherein the nucleic acid and compound are encapsulated in, bound to, or adsorbed on a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in water emulsion, a water-in-oil emulsion, an emulsome, a polycationic peptide, a cationic nanoemulsion, or a combination thereof.

E16. The composition of any one of embodiments E1 to E11, wherein the at least one nucleic acid and the at least one compound are encapsulated in a lipid nanoparticle (LNP), wherein said LNP comprises at least one phospholipid.

E17. The composition of embodiment E16 wherein the phospholipid is 1,2-distearoyl-sn-glycero-3-phosphocholine (DSPC).

E18. The composition of any one of embodiments E1 to E11, wherein the at least one nucleic acid and the at least one compound are encapsulated in a lipid nanoparticle (LNP), wherein said LNP comprises at least one steroid.

E19. The composition of embodiment E18, wherein the steroid is cholesterol.

E20. The composition of any one of embodiments E1 to E11, wherein the at least one nucleic acid and the at least one compound are encapsulated in a lipid nanoparticle (LNP), wherein said LNP comprises at least one cationic lipid.

E21. The composition of embodiment E20, wherein the at least one cationic lipid is [(4-hydroxybutyl)azanediyl]di(hexane-6,1-diyl) bis(2-hexyldecanoate).

E22. The composition of any one of embodiments E1 to E11, wherein the at least one nucleic acid and the at least one compound are encapsulated in a lipid nanoparticle (LNP), wherein said LNP comprises at least one PEG lipid.

E23. The composition of embodiment E22, wherein the at least one PEG lipid is 2-[(polyethylene glycol)-2000]-N,N-ditetradecylacetamide.

E24. The composition of any one of embodiments E1 to E23, wherein said at least one compound is a modulator of IRAK4.

E25. The composition of embodiment E24, wherein the at least one compound is selected from the group consisting of: zimlovisertib; 1-{[(2S,3S,4S)-4-fluoro-3-methyl-5-oxopyrrolidin-2-yl]methoxy}-7-methoxyisoquinoline-6-carboxamide; N-{3-(difluoromethyl)-1-[(1S,4R)-4-({4-[(3-{1-[(3S)-2,6-dioxopiperidin-3-yl]-3-methyl-2-oxo-2,3-dihydro-1H-benzimidazol-4-yl}prop-2-yn-1-yl)oxy]piperidin-1-yl}methyl)cyclohexyl]-1H-pyrazol-4-yl}-5-[(1R,4R)-2-oxa-5-azabicyclo[2,2,1]heptan-5-yl]pyrazolo[1,5-a]pyrimidine-3-carboxamide; and N-{2-[(1R,4r)-4-({[1-({1-[(3R)-2,6-dioxopiperidin-3-yl]-3-methyl-2-oxo-2,3-dihydro-1H-benzimidazol-4-yl}methyl)piperidin-4-yl](methyl)amino}methyl)cyclohexyl]-6-(2-hydroxypropan-2-yl)-2H-indazol-5-yl}-6-(trifluoromethyl)pyridine-2-carboxamide, or a pharmaceutically acceptable salt thereof.

E26. The composition of embodiment E25, wherein said compound is zimlovisertib, or a pharmaceutically acceptable salt thereof.

E27. The composition of embodiment E25, wherein said compound is 1-{[(2S,3S,4S)-4-fluoro-3-methyl-5-oxopyrrolidin-2-yl]methoxy}-7-methoxyisoquinoline-6-carboxamide, or a pharmaceutically acceptable salt thereof.

E28. The composition of embodiment E25, wherein said compound is N-{3-(difluoromethyl)-1-[(1S,4R)-4-({4-[(3-{1-[(3S)-2,6-dioxopiperidin-3-yl]-3-methyl-2-oxo-2,3-dihydro-1H-benzimidazol-4-yl}prop-2-yn-1-yl)oxy]piperidin-1-yl}methyl)cyclohexyl]-1H-pyrazol-4-yl}-5-[(1R,4R)-2-oxa-5-azabicyclo[2,2,1]heptan-5-yl]pyrazolo[1,5-a]pyrimidine-3-carboxamide, or a pharmaceutically acceptable salt thereof.

E29. The composition of any one of embodiments E1 to E23, where said at least one compound modulates the signaling activity of RIG-I.

E30. The composition of embodiment E29, wherein said compound is 2,2′-(piperazine-1,4-diyl)bis(6,7-dimethoxyquinazolin-4-amine), or a pharmaceutically acceptable salt thereof.

E31. The composition of embodiment E29, wherein said compound is 6,7-dimethoxy-2-[4-(2-methylpyrimidin-4-yl)piperazin-1-yl]quinazolin-4-amine, or a pharmaceutically acceptable salt thereof.

E32. A method of modulating the innate immune response to a nucleic acid, comprising administering at least one nucleic acid to a subject in need thereof in combination with at least one compound that effectively modulates the innate immune response to said nucleic acid.

E33. The method of embodiment E32, wherein the at least one nucleic acid is RNA.

E34. The method of embodiment E33, wherein the RNA is modRNA.

E35. The method of embodiment E33 wherein the RNA is saRNA.

E36. The method of embodiment E35, wherein the saRNA comprises at least one open reading frame that encodes a gene of interest.

E37. The method of embodiment E36, wherein the gene of interest is derived from influenza virus hemagglutinin (HA).

E38. The method of embodiment E35, wherein the saRNA further comprises a 10 second reading frame that encodes a second gene of interest.

E39. The method of embodiment E38, wherein said second gene of interest is derived from influenza virus neuraminidase (NA).

E40. The method of any one of embodiments E32 to E39 wherein the nucleotides are naturally-occurring.

E41. The method of any one of embodiments E35 to E39 wherein at least 10% of the total nucleotides in the saRNA have been replaced with modified or unnatural nucleotides.

E42. The method of embodiment E41, wherein the modified or unnatural nucleotides are selected from the group consisting of pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

E43. The method of any one of embodiments E32 to E42, further comprising at least one cationic lipid.

E44. The method of any one of embodiments E32 to E43, wherein the nucleic acid and compound are encapsulated in, bound to, or adsorbed on a cationic lipid.

E45. The method of any one of embodiments E32 to E44, further comprising a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, a polycationic peptide, or a cationic nanoemulsion.

E46. The method of any one of embodiments E32 to E45, wherein the nucleic acid and compound are encapsulated in, bound to, or adsorbed on a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in water emulsion, a water-in-oil emulsion, an emulsome, a polycationic peptide, a cationic nanoemulsion, or a combination thereof.

E47. The method of any one of embodiments E32 to E42, wherein the nucleic acid and the compound are encapsulated in a lipid nanoparticle (LNP), wherein said LNP comprises at least one phospholipid.

E48. The method of embodiment E47 wherein the phospholipid is 1,2-distearoyl-sn-glycero-3-phosphocholine (DSPC).

E49. The method of any one of embodiments E32 to E42, wherein the nucleic acid and the compound are encapsulated in a lipid nanoparticle (LNP), wherein said LNP comprises at least one steroid.

E50. The method of embodiment E49, wherein the steroid is cholesterol.

E51. The method of any one of embodiments E32 to E42, wherein the nucleic acid and the compound are encapsulated in a lipid nanoparticle (LNP), wherein said LNP comprises at least one cationic lipid.

E52. The method of embodiment E51, wherein the cationic lipid is [(4-hydroxybutyl)azanediyl]di(hexane-6,1-diyl) bis(2-hexyldecanoate).

E53. The method of any one of embodiments 32 to 42, wherein the nucleic acid and the compound are encapsulated in a lipid nanoparticle (LNP), wherein said LNP comprises at least one PEG lipid.

E54. The method of embodiment E53, wherein the PEG lipid is 2-[(polyethylene glycol)-2000]-N,N-ditetradecylacetamide.

E55. The method of embodiment E32, wherein the compound is a modulator of IRAK4.

E56. The method of embodiment E55, wherein the compound is selected from the group consisting of: zimlovisertib; 1-{[(2S,3S,4S)-4-fluoro-3-methyl-5-oxopyrrolidin-2-yl]methoxy}-7-methoxyisoquinoline-6-carboxamide; N-{3-(difluoromethyl)-1-[(1S,4R)-4-({4-[(3-{1-[(3S)-2,6-dioxopiperidin-3-yl]-3-methyl-2-oxo-2,3-dihydro-1H-benzimidazol-4-yl}prop-2-yn-1-yl)oxy]piperidin-1-yl}methyl)cyclohexyl]-1H-pyrazol-4-yl}-5-[(1R,4R)-2-oxa-5-azabicyclo[2,2,1]heptan-5-yl]pyrazolo[1,5-a]pyrimidine-3-carboxamide; and N-{2-[(1R,4r)-4-({[1-({1-[(3R)-2,6-dioxopiperidin-3-yl]-3-methyl-2-oxo-2,3-dihydro-1H-benzimidazol- 4-yl}methyl)piperidin-4-yl](methyl)amino}methyl)cyclohexyl]-6-(2-hydroxypropan-2-yl)-2H-indazol-5-yl}-6-(trifluoromethyl)pyridine-2-carboxamide, or a pharmaceutically acceptable salt thereof.

E57. The method of embodiment E56, wherein the compound is zimlovisertib, or a pharmaceutically acceptable salt thereof.

E58. The method of embodiment E56, wherein the compound is 1-{[(2S,3S,4S)-4-fluoro-3-methyl-5-oxopyrrolidin-2-yl]methoxy}-7-methoxyisoquinoline-6-carboxamide, or a pharmaceutically acceptable salt thereof.

E59. The method of embodiment E56, wherein the compound is N-{3-(difluoromethyl)-1-[(1S,4R)-4-({4-[(3-{1-[(3S)-2,6-dioxopiperidin-3-yl]-3-methyl-2-oxo-2,3-dihydro-1H-benzimidazol-4-yl}prop-2-yn-1-yl)oxy]piperidin-1-yl}methyl)cyclohexyl]-1H-pyrazol-4-yl}-5-[(1R,4R)-2-oxa-5-azabicyclo[2,2,1]heptan-5-yl]pyrazolo[1,5-a]pyrimidine-3-carboxamide, or a pharmaceutically acceptable salt thereof.

E60. The method of embodiment E32, wherein the compound is co-formulated with the nucleic acid.

E61. The method of embodiment E32, wherein the compound is administered either before or after the nucleic acid is administered.

E62. The method of embodiment E32, wherein the compound is administered before the nucleic acid is administered.

E63. The method of embodiment E32, wherein the compound is administered after the nucleic acid is administered.

E64. The method of embodiment E32, wherein the compound modulates the signaling activity of RIG-I.

E65. The method of embodiment E64, wherein the compound is 2,2′-(piperazine-1,4-diyl)bis(6,7-dimethoxyquinazolin-4-amine), or a pharmaceutically acceptable salt thereof.

E66. The method of embodiment E64, wherein the compound is 6,7-dimethoxy-2-[4-(2-methylpyrimidin-4-yl)piperazin-1-yl]quinazolin-4-amine, or a pharmaceutically acceptable salt thereof.

E67. The method of embodiment E64, wherein the compound is co-formulated with the at least one nucleic acid.

E68. The method of embodiment E64, wherein the compound is administered either before or after the at least one nucleic acid is administered.

E69. The method of embodiment E64, wherein the compound is administered before the at least one nucleic acid is administered.

E70. The method of embodiment E64, wherein the compound is administered after the at least one nucleic acid is administered.

E71. The composition of any one of embodiments E1 to E23, wherein said at least one compound is a compound shown in Table 1.

E72. The composition of any one of embodiments E1 to E23, wherein said at least one compound comprises a DPQ core.

E73. A compound, or pharmaceutically acceptable salt thereof, selected from any of the compounds as shown in Table 1.

E74. The method of any one of embodiments E32 to E54, wherein the compound is selected from any of the compounds in Table 1, or a pharmaceutically acceptable salt thereof.

E75. A method of increasing the expression of a protein encoded by a nucleic acid, comprising administering at least one nucleic acid to a subject in need thereof in combination with at least one compound that effectively increases the expression of said protein.

E76. The method of embodiment E75, wherein the at least one nucleic acid is RNA.

E77. The method of embodiment E76, wherein the RNA is modRNA.

E78. The method of embodiment E76 wherein the RNA is saRNA.

E79. The method of embodiment E78, wherein the saRNA comprises at least one open reading frame that encodes a gene of interest.

E80. The method of embodiment E79, wherein the gene of interest is derived from influenza virus hemagglutinin (HA).

E81. The method of embodiment E78, wherein the saRNA further comprises a second reading frame that encodes a second gene of interest.

E82. The method of embodiment E81, wherein said second gene of interest is derived from influenza virus neuraminidase (NA).

E83. The method of any one of embodiments E75 to E82 wherein the nucleotides are naturally-occurring.

E84. The method of any one of embodiments E78 to E82 wherein at least 10% of the total nucleotides in the saRNA have been replaced with modified or unnatural nucleotides.

E85. The method of embodiment E84, wherein the modified or unnatural nucleotides are selected from the group consisting of pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

E86. The method of any one of embodiments E75 to E85, further comprising at least one cationic lipid.

E87. The method of any one of embodiments E75 to E86, wherein the nucleic acid and compound are encapsulated in, bound to, or adsorbed on a cationic lipid.

E88. The method of any one of embodiments E75 to E87, further comprising a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, a polycationic peptide, or a cationic nanoemulsion.

E89. The method of any one of embodiments E75 to E88, wherein the nucleic acid and compound are encapsulated in, bound to, or adsorbed on a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in water emulsion, a water-in-oil emulsion, an emulsome, a polycationic peptide, a cationic nanoemulsion, or a combination thereof.

E90. The method of any one of embodiments E75 to E85, wherein the nucleic acid and the compound are encapsulated in a lipid nanoparticle (LNP), wherein said LNP comprises at least one phospholipid.

E91. The method of embodiment E89 wherein the phospholipid is 1,2-distearoyl-sn-glycero-3-phosphocholine (DSPC).

E92. The method of any one of embodiments E75 to E85, wherein the nucleic acid and the compound are encapsulated in a lipid nanoparticle (LNP), wherein said LNP comprises at least one steroid.

E93. The method of embodiment E92, wherein the steroid is cholesterol.

E94. The method of any one of embodiments E75 to E85, wherein the nucleic acid and the compound are encapsulated in a lipid nanoparticle (LNP), wherein said LNP comprises at least one cationic lipid.

E95. The method of embodiment E94, wherein the cationic lipid is [(4-hydroxybutyl)azanediyl]di(hexane-6,1-diyl) bis(2-hexyldecanoate).

E96. The method of any one of embodiments E75 to E85, wherein the nucleic acid and the compound are encapsulated in a lipid nanoparticle (LNP), wherein said LNP comprises at least one PEG lipid.

E97. The method of embodiment E96, wherein the PEG lipid is 2-[(polyethylene glycol)-2000]-N,N-ditetradecylacetamide.

E98. The method of embodiment E75, wherein the compound is a modulator of IRAK4.

E99. The method of embodiment E98, wherein the compound is selected from the group consisting of: zimlovisertib; 1-{[(2S,3S,4S)-4-fluoro-3-methyl-5-oxopyrrolidin-2-yl]methoxy}-7-methoxyisoquinoline-6-carboxamide; N-{3-(difluoromethyl)-1-[(1S,4R)-4-({4-[(3-{1-[(3S)-2,6-dioxopiperidin-3-yl]-3-methyl-2-oxo-2,3-dihydro-1H-benzimidazol-4-yl}prop-2-yn-1-yl)oxy]piperidin-1-yl}methyl)cyclohexyl]-1H-pyrazol-4-yl}-5-[(1R,4R)-2-oxa-5-azabicyclo[2,2,1]heptan-5-yl]pyrazolo[1,5-a]pyrimidine-3-carboxamide; and N-{2-[(1R,4r)-4-({[1-({1-[(3R)-2,6-dioxopiperidin-3-yl]-3-methyl-2-oxo-2,3-dihydro-1H-benzimidazol-4-yl}methyl)piperidin-4-yl](methyl)amino}methyl)cyclohexyl]-6-(2-hydroxypropan-2-yl)-2H-indazol-5-yl}-6-(trifluoromethyl)pyridine-2-carboxamide, or a pharmaceutically acceptable salt thereof.

E100. The method of embodiment E99, wherein the compound is zimlovisertib, or a pharmaceutically acceptable salt thereof.

E101. The method of embodiment E99, wherein the compound is 1-{[(2S,3S,4S)-4-fluoro-3-methyl-5-oxopyrrolidin-2-yl]methoxy}-7-methoxyisoquinoline-6-carboxamide, or a pharmaceutically acceptable salt thereof.

E102. The method of embodiment E99, wherein the compound is N-{3-(difluoromethyl)-1-[(1S,4R)-4-({4-[(3-{1-[(3S)-2,6-dioxopiperidin-3-yl]-3-methyl-2-oxo-2,3-dihydro-1H-benzimidazol-4-yl}prop-2-yn-1-yl)oxy]piperidin-1-yl}methyl)cyclohexyl]-1H-pyrazol-4-yl}-5-[(1R,4R)-2-oxa-5-azabicyclo[2,2,1]heptan-5-yl]pyrazolo[1,5-a]pyrimidine-3-carboxamide, or a pharmaceutically acceptable salt thereof.

E103. The method of embodiment E75, wherein the compound is co-formulated with the nucleic acid.

E104. The method of embodiment E75, wherein the compound is administered either before or after the nucleic acid is administered.

E105. The method of embodiment E75, wherein the compound is administered before the nucleic acid is administered.

E106. The method of embodiment E75, wherein the compound is administered after the nucleic acid is administered.

E107. The method of embodiment E75, wherein the compound modulates the signaling activity of RIG-I.

E108. The method of embodiment E103 or E104, wherein the compound is 2,2′-(piperazine-1,4-diyl)bis(6,7-dimethoxyquinazolin-4-amine), or a pharmaceutically acceptable salt thereof.

E109. The method of embodiment E103 or E104, wherein the compound is 6,7-dimethoxy-2-[4-(2-methylpyrimidin-4-yl)piperazin-1-yl]quinazolin-4-amine, or a pharmaceutically acceptable salt thereof.

E110. The method of any one of embodiments E74 to E106, wherein the compound is selected from any of the compounds in Table 1, or a pharmaceutically acceptable salt thereof.

E111. The composition of any one of embodiments E1 to E23, wherein said at least one compound is selected from the compounds provided in Table 1, or a pharmaceutically acceptable salt thereof.

E112. A composition comprising at least one nucleic acid and at least one compound selected from the compounds provided in Table 1, or a pharmaceutically acceptable salt thereof.

E113. The composition of embodiment E112, wherein the nucleic acid is according to any of the embodiments of E2 to E31.

E114. A method of delivering a nucleic acid to a subject in need thereof, comprising administering said nucleic acid to said subject in combination with at least one compound selected from any of the compounds as provided in Table 1.

E115. The method of embodiment E114, wherein the nucleic acid is according to any of the embodiments of E32 to E70.

E116. A method of delivering a nucleic acid to a subject in need thereof, comprising administering said nucleic acid to said subject in combination with at least one compound comprising a DPQ core.

E117. The method of embodiment E116, wherein the nucleic acid is according to any of the embodiments of E32 to E70.

### Definitions

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive “or.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The phrase “essentially all” is defined as “at least 95%”; if essentially all members of a group have a certain property, then at least 95% of members of the group have that property. In some instances, essentially all means equal to any one of, at least any one of, or between any two of 95, 96, 97, 98, 99, or 100% of members of the group have that property.

The compositions and methods for their use may “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed disclosure.

The term “RNA,” as used herein, means a sequence of nucleotides comprising a nucleotide base adenine (A), cytosine (C), guanine (G), and uracil (U) attached to a sugar such as ribose attached to a phosphate group wherein the nucleotide base, sugar, or phosphate may be modified wherein such modifications are known to those skilled in the art. It is to be understood that this definition includes single strand RNAs and double stranded RNAs.

The term “pharmaceutically acceptable salt,” as used herein, means those salts within the scope of sound medical judgement that are suitable for use in contact with the tissues of humans and animals without undue toxicity, irritation, allergic response and the like and are commensurate with a reasonable benefit/risk ratio. Pharmaceutically acceptable salts are well-known in the art. For example, S. M. Berge et al. describe pharmaceutically acceptable salts in detail in J. Pharmaceutical Sciences, 1977, 66:1-19, herein incorporated by reference. The salts can be prepared in situ during the final isolation and purification of the compounds of the present disclosure or separately by reacting a free base (basic nitrogen) with a suitable organic or inorganic acid. Representative acid addition salts include, but are not limited to acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid. The term “pharmaceutically acceptable salt,” as used herein, also means salts of carboxylic acid and sulfonic acid groups prepared by reacting the free acid with a positively charged inorganic or organic ion (cation) that is generally considered suitable for human consumption. Examples of pharmaceutically acceptable cations are lithium, sodium, potassium, magnesium, calcium, ferrous, ferric, ammonium, alkylammonium, dialkylammonium, trialkylammonium, tetraalkylammonium, diethanolammmonium, and choline. Cations may be interchanged by methods known in the art, such as ion exchange. Where compounds of the present disclosure contain one or more carboxylic acid groups or sulfonic acid groups, addition of a base (such as a hydroxide or a free amine) will yield the appropriate cationic form.

The term “modRNA” as used herein means a RNA that contains at least one modified, or unnatural, nucleotide.

The terms “saRNA” and “replicon” as used herein refer to RNA with the ability to replicate itself. Self-amplifying RNA molecules may be produced by using replication elements derived from, e.g., alphaviruses, and substituting the structural viral polypeptides with a nucleotide sequence encoding a polypeptide of interest.

The term “treating”, “treat” or “treatment” as used herein embraces both preventative, e.g., prophylactic, and palliative treatment, e.g., relieve, alleviate, or slow the progression of the patient's disease (or condition) or any tissue damage associated with the disease.

As used herein, the terms, “subject, “individual” or “patient,” used interchangeably, refer to any animal, including mammals. Mammals according to the disclosure include canine, feline, bovine, caprine, equine, ovine, porcine, rodents, lagomorphs, primates, humans and the like, and encompass mammals in utero. In an embodiment, humans are suitable subjects. Human subjects may be of any gender and at any stage of development.

As used herein, the phrase “therapeutically effective amount” or “effective amount” refers to the amount of active compound or pharmaceutical agent, such as a nucleic acid, that elicits the biological or medicinal response in a tissue, system, animal, individual or human that is being sought by a researcher, veterinarian, medical doctor or other clinician, which may include one or more of the following:
(1) preventing the disease; for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (e.g., arresting (or slowing) further development of the pathology or symptomatology or both); and
(3) ameliorating the disease; for example, ameliorating a disease, condition or disorder in an individual that is experiencing or displaying the pathology or symptomatology of the disease, condition or disorder (e.g., reversing the pathology or symptomatology or both).

As used herein, the term “modulate”, as well as words stemming therefrom, can mean effecting a change in the function or operation of a protein or a biological process. For example, such “modulation” can be either the upregulation or downregulation of the function of the target or process as compared to the function in the absence of the modulator. The term “modulate” can also be used to mean an upregulation or a downregulation of expression of a gene product as compared to the expression in the absence of the modulator. Furthermore, the term “modulate” can also mean to increase or decrease the endogenous activity of a protein in a cell, or in a cellular process as compared to such activity in the absence of the modulator. For example, the binding of compound to a protein, such as a receptor, for example, can alter the affinity of the receptor for binding its endogenous ligand, which can change how it interacts with other cellular proteins. Accordingly, as will be readily understood by those skilled in the art, the term “modulator” as used herein can mean a compound, or other biological substance that “modulates” the function of a given target or process as described above.

### Synthesis of Compounds

The small molecule compounds useful for the methods and compositions of the present disclosure were prepared using synthetic strategies known to those skilled in the art and by the synthetic strategies disclosed in, e.g. K. L. Lee et al., J. Med. Chem. 60:5521-5542 (2017); WO 2020/113233; U.S. Pat. No. 4,734,418; and European Patent Application No. 0055583A1, all of which are hereby incorporated by reference in their entirety.

Small molecule compounds useful in the methods and compositions of the present disclosure can exist as stereoisomers, wherein asymmetric or chiral centers are present. Stereoisomers are designated (R) or (S), depending on the configuration of substituents around the chiral carbon atom. The terms (R) and (S) used herein are configurations as defined in IUPAC 1974 Recommendations for Section E, Fundamental Stereochemistry, Pure Appl. Chem., (1976), 45: 13-30. The present disclosure contemplates various stereoisomers and mixtures thereof and are specifically included within the scope of this disclosure. Stereoisomers include enantiomers, diastereomers, and mixtures of enantiomers or diastereomers. Individual stereoisomers of compounds of the present disclosure may be prepared synthetically from commercially available starting materials which contain asymmetric or chiral centers or by preparation of racemic mixtures followed by resolution, a technique well-known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) direct separation of the mixture of optical enantiomers on chiral chromatographic columns, or (3) formation of a diastereomeric salt followed by selective recrystallization of one of the diastereomeric salts.

Small molecule compounds useful in the methods and compositions of the present disclosure include “deuterated analogues” of the compounds wherein from 1 to n hydrogens attached to a carbon atom is/are replaced by deuterium, wherein n is the number of hydrogens in the molecule. The deuterated compounds of the present disclosure are synthesized by means well known in the art, for example by employing starting materials in which one or more hydrogens have been replaced by deuterium.

Small molecule compounds useful in the methods and compositions of the present disclosure and disclosed in Table 1 were named by Chemdraw® Professional version 18.0.0.231 (4029) or were given names which appeared to be consistent with Chemdraw® nomenclature.

TABLE 1

| PF-Number | Structure | Nomenclature |
|---|---|---|
| PF-06650833 | 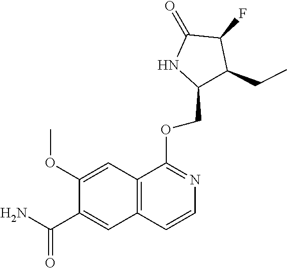 [SMILES: CC[C@@H]1[C@H](F)C(=O)N[C@@H]1COc1nccc2cc(C(N)=O)c(OC)cc12] | 1-{[(2S,3S,4S)-3-ethyl- 4-fluoro-5- oxopyrrolidin-2- yl]methoxy}-7- methoxyisoquinoline- 6-carboxamide (zimlovisertib) |
| PF-06426779 | 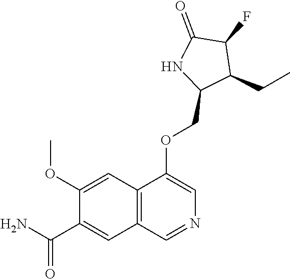 [SMILES: CC[C@@H]1[C@H](F)C(=O)N[C@@H]1COc1cncc2cc(C(N)=O)c(OC)cc12] | 1-{[(2S,3S,4S)-4- fluoro-3-methyl-5- oxopyrrolidin-2- yl]methoxy}-7- methoxyisoquinoline- 6-carboxamide |
| PF-07911620 | 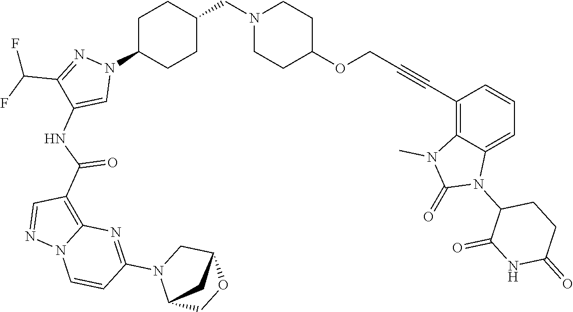 [SMILES: Cn1c(=O)n(C2CCC(=O)NC2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21] | N-{3-(difluoromethyl)- 1-[(1S,4R)-4-({4-[(3- {1-[(3S)-2,6- dioxopiperidin-3-yl]-3- methyl-2-oxo-2,3- dihydro-1H- benzimidazol-4- yl}prop-2-yn-1- yl)oxy]piperidin-1- yl}methyl)cyclohexyl]- 1H-pyrazol-4-yl}-5-[(1 R,4R)-2-oxa-5- azabicyclo[2,2,1]heptan- 5-yl]pyrazolo[1,5- a]pyrimidine-3- carboxamide |
| PF-07932784 | 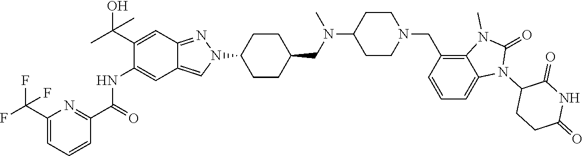 [SMILES: CN(C[C@H]1CC[C@H](n2cc3cc(NC(=O)c4cccc(C(F)(F)F)n4)c(C(C)(C)O)cc3n2)CC1)C1CCN(Cc2cccc3c2n(C)c(=O)n3C2CCC(=O)NC2=O)CC1] | N-{2-[(1R,4r)-4-({[1- ({1-[(3R)-2,6- dioxopiperidin-3-yl]-3- methyl-2-oxo-2,3- dihydro-1H- benzimidazol-4- yl}methyl)piperidin-4- yl](methyl)amino}meth yl)cyclohexyl]-6-(2- hydroxypropan-2-yl)- 2H-indazol-5-yl} -6- (trifluoromethyl)pyridine- 2-carboxamide |
| PF-01009329 | 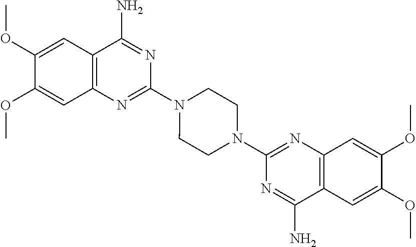 [SMILES: COc1cc2nc(N3CCN(c4nc(N)c5cc(OC)c(OC)cc5n4)CC3)nc(N)c2cc1OC] | 2,2′-(piperazine-1,4- diyl)bis(6,7- dimethoxyquinazolin- 4-amine) |
| PF-01034118 | 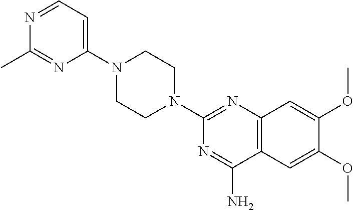 | 6,7-dimethoxy-2-[4-(2- methylpyrimidin-4- yl)piperazin-1- yl]quinazolin-4-amine |
| PF-01033181 | 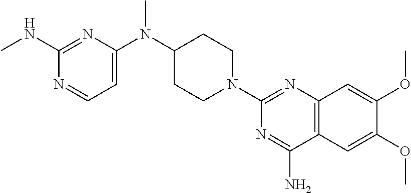 | N~4 ~- [1-(4-amino-6,7- dimethoxyquinazolin- 2-yl)piperidin-4-yl]- N~2~,N~4 ~- dimethylpyrimidine- 2,4-diamine |
| PF-01033811 | 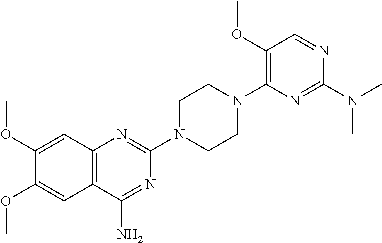 [SMILES: COc1cc2nc(N3CCN(c4nc(N(C)C)ncc4OC)CC3)nc(N)c2cc1OC] | 2-{4-[2- (dimethylamino)-5- methoxypyrimidin-4- yl]piperazin-1-yl}-6,7- dimethoxyquinazolin- 4-amine |
| PF-01034488 | 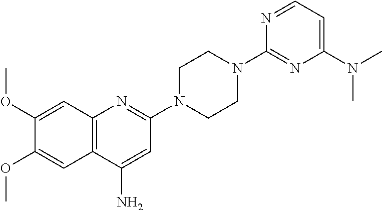 [SMILES: COc1cc2nc(N3CCN(c4nccc(N(C)C)n4)CC3)cc(N)c2cc1OC] | 2-{4-[4- (dimethylamino) pyrimidin-2-yl] piperazin-1-yl}-6,7- dimethoxyquinolin-4- amine |
| PF-01064258 | 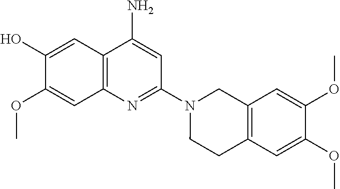 [SMILES: COc1cc2nc(N3CCc4cc(OC)c(OC)cc4C3)cc(N)c2cc1O] | 4-amino-2-(6,7- dimethoxy-3,4- dihydroisoquinolin- 2(1H)-yl)-7- methoxyquinolin-6-ol |
| PF-01169811 | 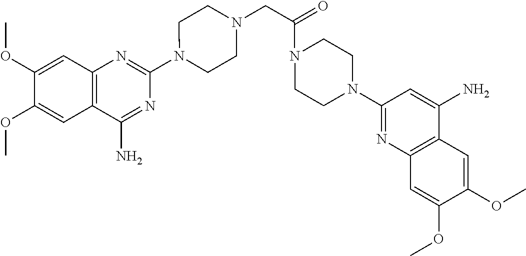 [SMILES: COc1cc2nc(N3CCN(C(=O)CN4CCN(c5nc(N)c6cc(OC)c(OC)cc6n5)CC4)CC3)cc(N)c2cc1OC] | 2-[4-(4-amino-6,7- dimethoxyquinazolin- 2-yl)piperazin-1-yl]-1- [4-(4-amino-6,7- dimethoxyquinolin-2- yl)piperazin-1- yl]ethan-1-one |
| PF-01027790 | 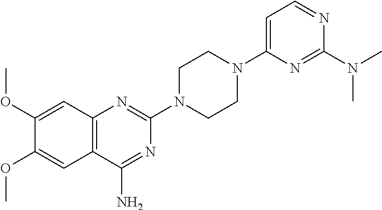 [SMILES: COc1cc2nc(N3CCN(c4ccnc(N(C)C)n4)CC3)nc(N)c2cc1OC] | 2-{4-[2- (dimethylamino)pyrimi din-4-yl]piperazin-1- yl}-6,7- dimethoxyquinazolin- 4-amine |
| PF-01027936 | 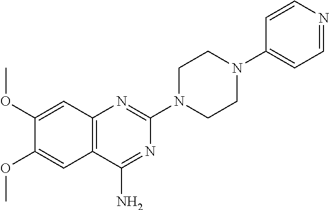 [SMILES: COc1cc2nc(N3CCN(c4ccncc4)CC3)nc(N)c2cc1OC] | 6,7-dimethoxy-2-[4- (pyridin-4-yl)piperazin- 1-yl]quinazolin-4- amine |
| PF-01033116 | 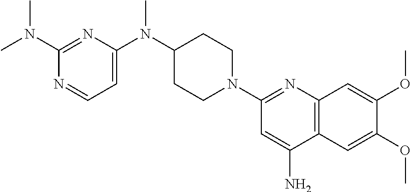 | N~4 ~- [1-(4-amino-6,7- dimethoxyquinolin-2- yl)piperidin-4-yl]- N~2~,N~2~,N~4 ~- trimethylpyrimidine- 2,4-diamine |
| PF-07299550 | 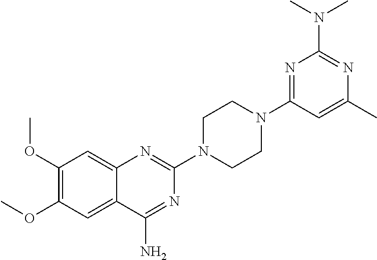 | 2-{4-[2- (dimethylamino)-6- methylpyrimidin-4- yl]piperazin-1-yl}-6,7- dimethoxyquinazolin- 4-amine |
| PF-07299558 | 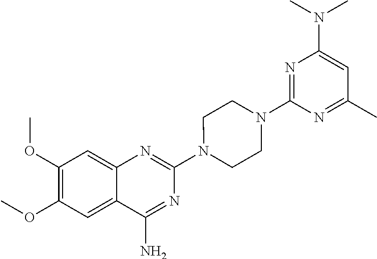 | 2-{4-[4- (dimethylamino)-6- methylpyrimidin-2- yl]piperazin-1-yl}-6,7- dimethoxyquinazolin- 4-amine |
| PF-07299608 | 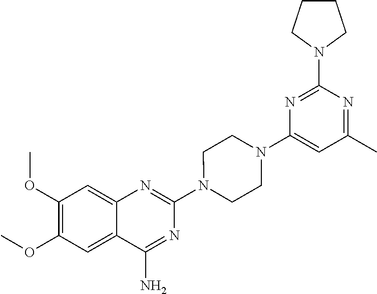 | 6,7-dimethoxy-2-{4-[6- methyl-2-(pyrrolidin-1- yl)pyrimidin-4- yl]piperazin-1- yl}quinazolin-4-amine |
| UK-040794 | 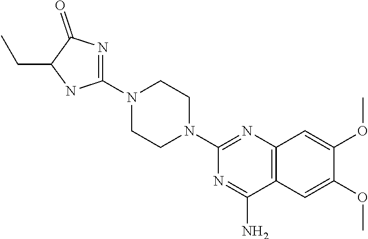 [SMILES: CCC1NC(N2CCN(c3nc(N)c4cc(OC)c(OC)cc4n3)CC2)=NC1=O] | 2-[4-(4-amino-6,7- dimethoxyquinazolin- 2-yl)piperazin-1-yl]-5- ethyl-1,3-thiazol- 4(5H)-one |
| PF-07299598 | 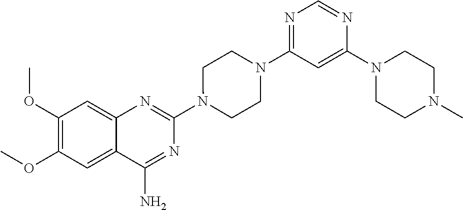 | 6,7-dimethoxy-2-{4-[6- (4-methylpiperazin-1- yl)pyrimidin-4- yl]piperazin-1- yl}quinazolin-4-amine |

### IRAK4 Modulators

Interleukin-1 receptor associated kinase 4 (IRAK4) is a ubiquitously expressed serine/threonine kinase involved in the regulation of innate immunity (Suzuki & Saito Trends in Immunology 27, 566 (2006)). IRAK4 is responsible for initiating signaling from Toll-like receptors (TLRs) and members of the IL-1/18 receptor family. Kinase-inactive knock-ins and targeted deletions of IRAK4 in mice were reported to cause reductions in TLR and IL-1 induced pro-inflammatory cytokines (Kawagoe et al., Journal of Experimental Medicine 204, 1013 (2007); Fraczek et al., Journal of Biological Chemistry 283, 31697 (2008); Kim et al., Journal of Experimental Medicine 204, 1025 (2007)). IRAK4 kinase-dead knock-in mice have also been shown to be resistant to induced joint inflammation in the antigen-induced-arthritis (AIA) and serum transfer-induced (K/BxN) arthritis models (Koziczak-Holbro et al., Arthritis & Rheumatism 60, 1661 (2009)). Likewise, humans deficient in IRAK4 also appear to display the inability to respond to challenge by Toll ligands and IL-1 (Hernandez & Bastian, Current Allergy and Asthma Reports 6, 468 (2006)). However, the immunodeficient phenotype of IRAK4-null individuals is narrowly restricted to challenge by gram positive bacteria, but not gram negative bacteria, viruses or fungi. This gram-positive sensitivity also lessens with age, implying redundant or compensating mechanisms for innate immunity in the absence of IRAK4 (Lavine et al., Journal of Allergy and Clinical Immunology 120, 948 (2007)). Accordingly, small molecule compounds that modulate the kinase activity of IRAK4 are potential therapeutics for a wide variety of diseases including but not limited to autoimmunity, inflammation, cardiovascular diseases, cancer, and metabolic diseases. IRAK4 “degraders” are bifunctional compounds that bind IRAK4 with one arm and the other consists of an immunomodulatory imide (IMiD) that recruits the E3 ligase complex component cereblon. A ubiquitin E3 ligase is an enzyme involved in marking proteins for ubiquitination and subsequent destruction by the proteasome. Such compounds are disclosed in references such as WO2015/150995, which is incorporated by reference in its entirety.

### RIG-1 Modulators

Retinoic acid-inducible gene I (RIG-1) is a cytoplasmic pathogen recognition receptor that plays a key role in the recognition of viruses, in particular RNA viruses. Once activated, RIG-1 leads a signaling cascade that activates IRF3 and NF-κB, which induces the expression of antiviral gene products and the production of interferons that results in an antiviral state in the infected cell (A. M. Kell et al., Virology Vol. 479-480, pp. 110-121 (2015)). Accordingly, small molecule compounds that modulate this receptor are potential therapeutics for a wide variety of diseases including but not limited to autoimmunity, inflammation, cardiovascular diseases, cancer, and metabolic diseases, such as diabetes. Such compounds are disclosed in references such as European Patent Application No. 0055583A1 and U.S. Pat. No. 4,734,418, which are incorporated by reference in their entirety.

### RNA

Nucleic acid sequences can exist in a variety of instances such as: isolated segments and recombinant vectors of incorporated sequences or recombinant polynucleotides encoding polypeptides, such as antigens or one or both chains of an antibody, or a fragment, derivative, mutein, or variant thereof; polynucleotides sufficient for use as hybridization probes; PCR primers or sequencing primers for identifying, analyzing, mutating or amplifying a polynucleotide encoding a polypeptide; anti-sense nucleic acids for inhibiting expression of a polynucleotide; mRNA, saRNA, and complementary sequences of the foregoing described herein; nucleic acids that encode an epitope to which antibodies may bind. The nucleic acids can be single-stranded or double-stranded and can comprise RNA and/or DNA nucleotides and artificial variants thereof (e.g., peptide nucleic acids).

The term “polynucleotide” refers to a nucleic acid molecule that can be recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar polypeptide.

In certain embodiments, there are polynucleotide variants having substantial identity to the sequences disclosed herein; those comprising equal to any one of, at least any one of, at most any one of, or between any two of 70%, 75%, 80%, 85%, 90%, 95%, 96%, 97%, 98%, or 99% or higher sequence identity, compared to a polynucleotide sequence provided herein using the methods described herein (e.g., BLAST analysis using standard parameters). In certain aspects, the isolated polynucleotide will comprise a nucleotide sequence encoding a polypeptide that has at least 90% identity to an amino acid sequence described herein, over the entire length of the sequence; or a nucleotide sequence complementary to said isolated polynucleotide. In some embodiments, the isolated polynucleotide will comprise a nucleotide sequence encoding a polypeptide that has at least 95% identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, equal to any one of, at least any one of, at most any one of, or between any two of 5, 10, 15, 20, 25, 30, 35, 40, 45, 50, 75, 100, 125, 175, 200, 250, 300, 350, 400, 450, 500, 750, 1000, 1500, 3000, 5000, 6000, 7000, 8000, 9000, 10000, 11000, 12000, 13000, 14000, 15000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

Self-Amplifying RNA (saRNA)

In some embodiments, the RNA molecule is an saRNA. “saRNA,” “self-amplifying RNA,” and “replicon” refer to RNA with the ability to replicate itself. Self-amplifying RNA molecules may be produced by using replication elements derived from a virus or viruses, e.g., alphaviruses, and substituting the structural viral polypeptides with a nucleotide sequence encoding a polypeptide of interest. A self-amplifying RNA molecule is typically a positive-strand molecule that may be directly translated after delivery to a cell, and this translation provides an RNA-dependent RNA polymerase which then produces both antisense and sense transcripts from the delivered RNA. The delivered RNA leads to the production of multiple daughter RNAs. These daughter RNAs, as well as collinear subgenomic transcripts, may be translated themselves to provide in situ expression of an encoded gene of interest, e.g., a viral antigen, or may be transcribed to provide further transcripts with the same sense as the delivered RNA which are translated to provide in situ expression of the protein of interest, e.g., an antigen. The overall result of this sequence of transcriptions is an amplification in the number of the introduced saRNAs and so the encoded gene of interest, e.g., a viral antigen, can become a major polypeptide product of the cells.

In some embodiments, the self-amplifying RNA includes at least one or more genes selected from any one of viral replicases, viral proteases, viral helicases and other nonstructural viral proteins. In some embodiments, the self-amplifying RNA may also include 5′- and 3′-end tractive replication sequences, and optionally a heterologous sequence that encodes a desired amino acid sequence (e.g., an antigen of interest). A subgenomic promoter that directs expression of the heterologous sequence may be included in the self-amplifying RNA. Optionally, the heterologous sequence (e.g., an antigen of interest) may be fused in frame to other coding regions in the self-amplifying RNA and/or may be under the control of an internal ribosome entry site (IRES).

In some embodiments, the self-amplifying RNA molecule is not encapsulated in a virus-like particle. Self-amplifying RNA molecules described herein may be designed so that the self-amplifying RNA molecule cannot induce production of infectious viral particles. This may be achieved, for example, by omitting one or more viral genes encoding structural proteins that are necessary to produce viral particles in the self-amplifying RNA. For example, when the self-amplifying RNA molecule is based on an alphavirus, such as Sinbis virus (SIN), Semliki forest virus and Venezuelan equine encephalitis virus (VEE), one or more genes encoding viral structural proteins, such as capsid and/or envelope glycoproteins, may be omitted.

In some embodiments, a self-amplifying RNA molecule described herein encodes (i) an RNA-dependent RNA polymerase that may transcribe RNA from the self-amplifying RNA molecule and (ii) a polypeptide of interest, e.g., a viral antigen. In some embodiments, the polymerase may be an alphavirus replicase, e.g., including any one of alphavirus protein nsP1, nsP2, nsP3, nsP4, and any combination thereof. In some embodiments, the self-amplifying RNA molecules described herein may include one or more modified nucleotides (e.g., pseudouridine, N6-methyladenosine, 5-methylcytidine, 5-methyluridine). In some embodiments, the self-amplifying RNA molecules does not include a modified nucleotide (e.g., pseudouridine, N6-methyladenosine, 5-methylcytidine, 5-methyluridine).

The saRNA construct may encode at least one non-structural protein (NSP), disposed 5′ or 3′ of the sequence encoding at least one peptide or polypeptide of interest. In some embodiments, the sequence encoding at least one NSP is disposed 5′ of the sequences encoding the peptide or polypeptide of interest. Thus, the sequence encoding at least one NSP may be disposed at the 5′ end of the RNA construct. In some embodiments, at least one non-structural protein encoded by the RNA construct may be the RNA polymerase nsP4. In some embodiments, the saRNA construct encodes nsP1, nsP2, nsP3 and, nsP4. As is known in the art, nsP1 is the viral capping enzyme and membrane anchor of the replication complex (RC). nsP2 is an RNA helicase and the protease responsible for the ns polyprotein processing. nsP3 interacts with several host proteins and may modulate protein poly- and mono-ADP-ribosylation. nsP4 is the core viral RNA-dependent RNA polymerase.

Whereas natural alphavirus genomes encode structural virion proteins in addition to the non-structural replicase polypeptide, in some embodiments, the self-amplifying RNA molecules do not encode alphavirus structural proteins. In some embodiments, the self-amplifying RNA may lead to the production of genomic RNA copies of itself in a cell, but not to the production of RNA that includes virions. Without being bound by theory or mechanism, the inability to produce these virions means that, unlike a wild-type alphavirus, the self-amplifying RNA molecule cannot perpetuate itself in infectious form. The alphavirus structural proteins which are necessary for perpetuation in wild-type viruses can be absent from self-amplifying RNAs of the present disclosure and their place can be taken by gene(s) encoding the immunogen of interest, such that the subgenomic transcript encodes the immunogen rather than the structural alphavirus virion proteins.

In some embodiments, the self-amplifying RNA molecule may have two open reading frames. The first (5) open reading frame can encode a replicase; the second (3′) open reading frame can encode a polypeptide comprising an antigen of interest. In some embodiments the RNA may have additional (e.g., downstream) open reading frames, e.g., to encode further antigens or to encode accessory polypeptides.

In some embodiments, the second RNA or the saRNA molecule further includes (1) an alphavirus 5′ replication recognition sequence, and (2) an alphavirus 3′ replication recognition sequence. In some embodiments, the 5′ sequence of the self-amplifying RNA molecule is selected to ensure compatibility with the encoded replicase.

Optionally, self-amplifying RNA molecules described herein may also be designed to induce production of infectious viral particles that are attenuated or virulent, or to produce viral particles that are capable of a single round of subsequent infection.

In some embodiments, the saRNA molecule is alphavirus-based. Alphaviruses include a set of genetically, structurally, and serologically related arthropod-borne viruses of the Togaviridae family. Exemplary viruses and virus subtypes within the alphavirus genus include Sindbis virus, Semliki Forest virus, Ross River virus, and Venezuelan equine encephalitis virus. As such, the self-amplifying RNA described herein may incorporate an RNA replicase derived from any one of semliki forest virus (SFV), sindbis virus (SIN), Venezuelan equine encephalitis virus (VEE), Ross-River virus (RRV), or other viruses belonging to the alphavirus family. In some embodiments, the self-amplifying RNA described herein may incorporate sequences derived from a mutant or wild-type virus sequence, e.g., the attenuated TC83 mutant of VEEV has been used in saRNAs.

Alphavirus-based saRNAs are (+)-stranded saRNAs that may be translated after delivery to a cell, which leads to translation of a replicase (or replicase-transcriptase). The replicase is translated as a polyprotein which auto-cleaves to provide a replication complex which creates genomic (−)-strand copies of the (+)-strand delivered RNA. These (−)-strand transcripts may themselves be transcribed to give further copies of the (+)-stranded parent RNA and also to give a subgenomic transcript which encodes the desired gene product. Translation of the subgenomic transcript thus leads to in situ expression of the desired gene product by the infected cell. Suitable alphavirus saRNAs may use a replicase from a sindbis virus, a semliki forest virus, an eastern equine encephalitis virus, a Venezuelan equine encephalitis virus, or mutant variants thereof.

In some embodiments, the self-amplifying RNA molecule is derived from or based on a virus other than an alphavirus, such as a positive-stranded RNA virus, and in particular a picornavirus, flavivirus, rubivirus, pestivirus, hepacivirus, calicivirus, or coronavirus. Suitable wild-type alphavirus sequences are well-known and are available from sequence depositories, such as the American Type Culture Collection, Rockville, Md. Representative examples of suitable alphaviruses include Aura (ATCC VR-368), Bebaru virus (ATCC VR-600, ATCC VR-1240), Cabassou (ATCC VR-922), Chikungunya virus (ATCC VR-64, ATCC VR-1241), Eastern equine encephalomyelitis virus (ATCC VR-65, ATCC VR-1242), Fort Morgan (ATCC VR-924), Getah virus (ATCC VR-369, ATCC VR-1243), Kyzylagach (ATCC VR-927), Mayaro (ATCC VR-66), Mayaro virus (ATCC VR-1277), Middleburg (ATCC VR-370), Mucambo virus (ATCC VR-580, ATCC VR-1244), Ndumu (ATCC VR-371), Pixuna virus (ATCC VR-372, ATCC VR-1245), Ross River virus (ATCC VR-373, ATCC VR-1246), Semliki Forest (ATCC VR-67, ATCC VR-1247), Sindbis virus (ATCC VR-68, ATCC VR-1248), Tonate (ATCC VR-925), Triniti (ATCC VR-469), Una (ATCC VR-374), Venezuelan equine encephalomyelitis (ATCC VR-69, ATCC VR-923, ATCC VR-1250 ATCC VR-1249, ATCC VR-532), Western equine encephalomyelitis (ATCC VR-70, ATCC VR-1251, ATCC VR-622, ATCC VR-1252), Whataroa (ATCC VR-926), and Y-62-33 (ATCC VR-375).

In some embodiments, the self-amplifying RNA molecules described herein are larger than other types of RNA. Typically, the self-amplifying RNA molecules described herein include at least about 4 kb. For example, the self-amplifying RNA may be equal to any one of, at least any one of, at most any one of, or between any two of 3 kb, 4 kb, 5 kb, 6 kb, 7 kb, 8 kb, 9 kb, 10 kb, 11 kb, 12 kb, 13 kb, 14 kb, 15 kb, 16 kb. In some instances the self-amplifying RNA may include at least about 5 kb, at least about 6 kb, at least about 7 kb, at least about 8 kb, at least about 9 kb, at least about 10 kb, at least about 11 kb, at least about 12 kb, or more than 12 kb. In certain examples, the self-amplifying RNA is about 4 kb to about 12 kb, about 5 kb to about 12 kb, about 6 kb to about 12 kb, about 7 kb to about 12 kb, about 8 kb to about 12 kb, about 9 kb to about 12 kb, about 10 kb to about 12 kb, about 11 kb to about 12 kb, about 5 kb to about 11 kb, about 5 kb to about 10 kb, about 5 kb to about 9 kb, about 5 kb to about 8 kb, about 5 kb to about 7 kb, about 5 kb to about 6 kb, about 6 kb to about 12 kb, about 6 kb to about 11 kb, about 6 kb to about 10 kb, about 6 kb to about 9 kb, about 6 kb to about 8 kb, about 6 kb to about 7 kb, about 7 kb to about 11 kb, about 7 kb to about 10 kb, about 7 kb to about 9 kb, about 7 kb to about 8 kb, about 8 kb to about 11 kb, about 8 kb to about 10 kb, about 8 kb to about 9 kb, about 9 kb to about 11 kb, about 9 kb to about 10 kb, or about 10 kb to about 11 kb.

In some embodiments, the self-amplifying RNA molecule may encode a single polypeptide antigen or, optionally, two or more polypeptide antigens linked together in a way that each of the sequences retains its identity (e.g., linked in series) when expressed as an amino acid sequence. The polypeptides generated from the self-amplifying RNA may then be produced as a fusion polypeptide or engineered in such a manner to result in separate polypeptide sequences. In some embodiments, the saRNA molecule may encode, e.g., two, three, four, five, six, seven, eight, nine, ten, or more polypeptides. Alternatively, or in addition, one saRNA molecule may also encode more than one polypeptide of interest or more, such as an antigen, e.g., a bicistronic, or tricistronic RNA molecule that encodes different or identical antigens.

In some embodiments, the self-amplifying RNA described herein may encode one or more polypeptide antigens that include a range of epitopes. In some embodiments, the self-amplifying RNA described herein may encode epitopes capable of eliciting either a helper T-cell response or a cytotoxic T-cell response or both.

In some embodiments, the saRNA molecule is purified, e.g., such as by filtration that may occur via, e.g., ultrafiltration, diafiltration, or, e.g., tangential flow ultrafiltration/diafiltration.

Some embodiments of the disclosure are directed to a composition comprising a saRNA molecule comprising a 5′ Cap, a 5′ untranslated region, a coding region comprising a sequence encoding an RNA-dependent RNA polymerase (also referred to as a “replicase”), a subgenomic promoter, such as one derived from an alphavirus, an open reading frame encoding a gene of interest (e.g., an antigen derived from influenza virus), a 3′ untranslated region, and a 3′ poly A sequence. In some embodiments, at least 5% of a total population of a particular nucleotide in the saRNA molecule has been replaced with one or more modified or unnatural nucleotides.

In some embodiments, the saRNA molecule does not include modified nucleotides, e.g., does not include modified nucleobases, and all of the nucleotides in the RNA molecule are conventional standard ribonucleotides A, U, G and C, with the exception of an optional 5′ cap that may include, for example, 7-methylguanosine, which is further described below. In some embodiments, the RNA may include a 5′ cap comprising a 7′-methylguanosine, and the first 1, 2 or 3 5′ ribonucleotides may be methylated at the 2′ position of the ribose.

The efficacy of the product is dependent on expression of the delivered saRNA, which requires a sufficiently intact RNA molecule. RNA integrity is a measure of RNA quality that quantitates intact RNA. RNA integrity is preferably determined by capillary gel electrophoresis. The initial specification is set to ensure sufficient RNA integrity in drug product preparations. In some embodiments, the RNA polynucleotide has an integrity of at least about 80%, 85%, 90%, 92%, 94%, 95%, 96%, 97%, 98%, or 99%. In some embodiments, the RNA polynucleotide has an integrity of or greater than about 95%. In some embodiments, the RNA polynucleotide has an integrity of or greater than about 98%. In some embodiments, the RNA polynucleotide has an integrity of or greater than about 99%.

In preferred embodiments, the saRNA has a clinical grade purity. In some embodiments, the purity of the saRNA is between about 60% and about 100%. In some embodiments, the purity of the saRNA is between about 80% and 99%. In some embodiments, the purity of the saRNA is between about 90% and about 99%. In some embodiments, the purified saRNA has a clinical grade purity without further purification. In some embodiments, the clinical grade purity is achieved through a method including tangential flow filtration (TFF) purification. In some embodiments, the clinical grade purity is achieved without the further purification selected from high performance liquid chromatography (HPLC) purification, ligand or binding based purification, and/or ion exchange chromatography. In some embodiments, the method of producing the RNA polynucleotides removes long abortive RNA species, double-stranded RNA (dsRNA), residual plasmid DNA residual solvent and/or residual salt. In some embodiments, the short abortive transcript contaminants comprise less than 15 bases. In some embodiments, the short abortive transcript contaminants comprise about 8 to 12 bases. In some embodiments, the method of the disclosure also removes RNAse inhibitor.

In some embodiments, the purified saRNA comprises 5% or less, 4% or less, 3% or less, 2% or less, 1% or less or is substantially free of protein contaminants as determined by capillary electrophoresis. In some embodiments, the purified saRNA comprises less than 5%, less than 4%, less than 3%, less than 2%, less than 1%, or is substantially free of salt contaminants determined by high performance liquid chromatography (HPLC). In some embodiments, the purified saRNA comprises 5% or less, 4% or less, 3% or less, 2% or less, 1% or less or is substantially free of short abortive transcript contaminants determined by known methods, such as, e.g., high performance liquid chromatography (HPLC). In some embodiments, the purified saRNA has integrity of 60% or greater, 70% or greater, 80% or greater, 81% or greater, 82% or greater, 83% or greater, 84% or greater, 85% or greater, 86% or greater, 87% or greater, 88% or greater, 89% or greater, 90% or greater, 95% or greater, 96% or greater, 97% or greater, 98% or greater, or 99% or greater as determined by a known method, such as, e.g., capillary electrophoresis.

### Modified Nucleobases

Modified nucleobases which may be incorporated into modified nucleosides and nucleotides and be present in the RNA molecules (i.e. “modRNA”) of the present disclosure include, for example, m5C (5-methylcytidine), m5U (5-methyluridine), m6A (N6-methyladenosine), s2U (2-thiouridine), Um (2′-0-methyluridine), mIA (1-methyladenosine); m2A (2-methyladenosine); Am (2-1-O-methyladenosine); ms2m6A (2-methylthio-N6-methyladenosine); i6A (N6-isopentenyladenosine); ms2i6A (2-methylthio-N6isopentenyladenosine); io6A (N6-(cis-hydroxyisopentenyl)adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A(N6-hydroxynorvalylcarbamoyl adenosine); ms2hn6A (2-methylthio-N6-hydroxynorvalyl carbamoyladenosine); Ar(p) (2′-0-ribosyladenosine (phosphate)); I (inosine); mil (1-methylinosine); m'Im (1,2′-0-dimethylinosine); m3C (3-methylcytidine); Cm (2T-0-methylcytidine); s2C (2-thiocytidine); ac4C (N4-acetylcytidine); £5C (5-fonnylcytidine); m5Cm (5,2-0-dimethylcytidine); ac4Cm (N4acetyl2TOmethylcytidine); k2C (lysidine); mIG (1-methylguanosine); m2G (N2-methylguanosine); m7G (7-methylguanosine); Gm (2′-0-methylguanosine); m22G (N2,N2-dimethylguanosine); m2Gm (N2,2′-0-dimethylguanosine); m22Gm (N2,N2,2′-0-trimethylguanosine); Gr(p) (2′-0-ribosylguanosine (phosphate)); yW (wybutosine); o2yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-0-dimethyluridine); s4U (4-thiouridine); m5s2U (5-methyl-2-thiouridine); s2Um (2-thio-2′-O-methyluridine); acp3U (3-(3-amino-3-carboxypropyl)uridine); ho5U (5-hydroxyuridine); mo5U (5-methoxyuridine); cmo5U (uridine 5-oxyacetic acid); mcmo5U (uridine 5 -oxyacetic acid methyl ester); chm5U (5-(carboxyhydroxymethyl)uridine)); mchm5U (5-(carboxyhydroxymethyl)uridine methyl ester); mcm5U (5-methoxycarbonyl methyluridine); mcm5Um (S-methoxycarbonylmethyl-2-O-methyluridine); mcm5s2U (5-methoxycarbonylmethyl-2-thiouridine); nm5s2U (5-aminomethyl-2-thiouridine); mnm5U (5-methylaminomethyluridine); mnm5s2U (5-methylaminomethyl-2-thiouridine); mnm5se2U (5-methylaminomethyl-2-selenouridine); ncm5U (5-carbamoylmethyl uridine); ncm5Um (5-carbamoylmethyl-2′-0-methyluridine); cmnm5U (5-carboxymethylaminomethyluridine); cnmm5Um (5-carboxymethy 1 aminomethyl-2-L-Omethyluridine); cmnm5s2U (5-carboxymethylaminomethyl-2-thiouridine); m62A (N6,N6-dimethyladenosine); Tm (2′-0-methylinosine); m4C (N4-methylcytidine); m4Cm (N4,2-0-dimethylcytidine); hm5C (5-hydroxymethylcytidine); m3U (3-methyluridine); cm5U (5-carboxymethyluridine); m6Am (N6,T-0-dimethyladenosine); rn62Am (N6,N6,0-2-trimethyladenosine); m2′7G (N2,7-dimethylguanosine); m2′2′7G (N2,N2,7-trimethylguanosine); m3Um (3,2T-0-dimethyluridine); m5D (5-methyldihydrouridine); f5Cm (5-formyl-2′-0-methylcytidine); mlGm (1,2′-0-dimethylguanosine); m'Am (1,2-0-dimethyl adenosine) irinomethyluridine); tm5s2U (S-taurinomethyl-2-thiouridine)); imG-14 (4-demethyl guanosine); imG2 (isoguanosine); ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(Ci-C6)-alkyluracil, 5-methyluracil, 5-(C2-Ce)-alkenyluracil, 5-(C2-Ce)-alkynyluracil, 5-(hydroxymethyl)uracil, 5-chlorouracil, 5-fluorouracil, 5-bromouracil, 5-hydroxycytosine, 5-(Ci-C6)-alkylcytosine, 5-methylcytosine, 5-(C2-C6)-alkenylcytosine, 5-(C2-C6)-alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), m5C, m5U, m6A, s2U, W, or 2′-O-methyl-U. In some aspects, one or more of the modified nucleosides in the list may be excluded.

Additional exemplary modified nucleotides include any one of N-1-methylpseudouridine; pseudouridine, N6-methyladenosine, 5-methylcytidine, and 5-methyluridine. In some embodiments, the modified nucleotide is N-1-methylpseudouridine.

In some embodiments, the RNA molecule may include phosphoramidate, phosphorothioate, and/or methylphosphonate linkages.

In some embodiments, at least 10% of a total population of a particular nucleotide in the RNA molecule has been replaced with one or more modified or unnatural nucleotides. In some embodiments, at least 25% of a total population of a particular nucleotide in the molecule has been replaced with one or more modified or unnatural nucleotides. In some embodiments, at least 50% of a total population of a particular nucleotide in the molecule has been replaced with one or more modified or unnatural nucleotides. In some embodiments, at least 75% of a total population of a particular nucleotide in the molecule has been replaced with one or more modified or unnatural nucleotides. In some embodiments, essentially all of the particular nucleotide population in the molecule has been replaced with one or more modified or unnatural nucleotides.

More particularly, in some embodiments, at least 25% of a total population of uridine nucleotides in the RNA molecule has been replaced with N1-methylpseudouridine. In some embodiments, at least 50% of a total population of uridine nucleotides in the molecule has been replaced with N1-methylpseudouridine. In some embodiments, at least 75% of a total population of uridine nucleotides in the molecule has been replaced with N1-methylpseudouridine. In some embodiments, essentially all uridine nucleotides in the molecule have been replaced with N1-methylpseudouridine. In some embodiments, at least 50% of a total population of uridine nucleotides in the molecule has been replaced with 5-methoxyuridine. In some embodiments, essentially all uridine nucleotides in the molecule have been replaced with 5-methoxyuridine. In some embodiments, at least 50% of a total population of uridine nucleotides in the molecule has been replaced with 5-methyluridine. In some embodiments, essentially all uridine nucleotides in the molecule have been replaced with 5-methyluridine. In some embodiments, at least 50% of a total population of cytosine nucleotides in the molecule has been replaced with 5-methylcytosine. In some embodiments, essentially all cytosine nucleotides in the molecule have been replaced with 5-methylcytosine. In some embodiments, at least 50% of a total population of uridine nucleotides in the molecule has been replaced with 2-thiouridine. In some embodiments, essentially all uridine nucleotides in the molecule have been replaced with 2-thiouridine.

Further, in some embodiments, at least 50% of a total population of uridine nucleotides in the molecule has been replaced with N1-methylpseudouridine and essentially all cytosine nucleotides in the molecule have been replaced with 5 -methylcytosine. In some embodiments, at least 50% of a total population of uridine nucleotides in the molecule has been replaced with 5-methoxyuridine and essentially all cytosine nucleotides in the molecule have been replaced with 5-methylcytosine. In some embodiments, at least 50% of a total population of uridine nucleotides in the molecule has been replaced with 5-methyluridine and essentially all cytosine nucleotides in the molecule have been replaced with 5-methylcytosine.

Even further, in some embodiments, essentially all uridine nucleotides in the molecule have been replaced with about 50% 5-methoxyuridine and about 50% N1-methylpseudouridine. In some embodiments, essentially all uridine nucleotides in the molecule have been replaced with about 75% 5-methoxyuridine and about 25% N1-methylpseudouridine. In some embodiments, essentially all uridine nucleotides in the molecule have been replaced with about 25% 5-methoxyuridine and about 75% N1-methylpseudouridine.

### Untranslated Regions (UTRs)

The 5′ untranslated region (UTR) is a regulatory region of DNA situated at the 5′ end of a protein coding sequence that is transcribed into mRNA but not translated into protein. 5′ UTRs may contain various regulatory elements, e.g., a 5′ cap structure, stem-loop structure, and an internal ribosome entry site (IRES), which may play a role in the control of translation initiation. The 3′ UTR, situated downstream of a protein coding sequence, may be involved in regulatory processes including transcript cleavage, stability and polyadenylation, translation, and mRNA localization. In some embodiments, the UTR increases protein synthesis. Without being bound by mechanism or theory, the UTR may increase protein synthesis by increasing the time that the mRNA remains in translating polysomes (message stability) and/or the rate at which ribosomes initiate translation on the message (message translation efficiency). Accordingly, the UTR sequence may prolong protein synthesis in a tissue-specific manner. In some embodiments, the 5′ UTR and the 3′ UTR sequences are computationally derived. In some embodiments, the 5′ UTR and the 3′ UTRs are derived from a naturally abundant mRNA in a tissue. The tissue may be, for example, liver, a stem cell, or lymphoid tissue. The lymphoid tissue may include, for example, any one of a lymphocyte (e.g., a B-lymphocyte, a helper T-lymphocyte, a cytotoxic T-lymphocyte, a regulatory T-lymphocyte, or a natural killer cell), a macrophage, a monocyte, a dendritic cell, a neutrophil, an eosinophil and a reticulocyte. In some embodiments, the 5′ UTR and the 3′ UTR are derived from an alphavirus. In some embodiments, the 5′ UTR and the 3′ UTR are from a wild-type alphavirus.

### Open Reading Frame (ORF)

The 5′ and 3′ UTRs may be operably linked to an ORF, which may be a sequence of codons that is capable of being translated into a polypeptide of interest. As stated above, the RNA molecule may include one (monocistronic), two (bicistronic) or more (multicistronic) open reading frames.

In some embodiments, the ORF encodes a non-structural viral gene. In some embodiments, the ORF further includes one or more subgenomic promoters. In some embodiments, the RNA molecule includes a subgenomic promoter operably linked to the ORF. In some embodiments, the subgenomic promoter comprises a cis-acting regulatory element. In some embodiments, the cis-acting regulatory element is immediately downstream (5′-3′) of B². In some embodiments, the cis-acting regulatory element is immediately downstream (5′-3′) of a guanine that is immediately downstream of B². In some embodiments, the cis-acting regulatory element is an AU-rich element. In some embodiments, the AU-rich element is au, auaaaagau, auaaaaagau, auag, auauauauau, auauauau, auauauauauau, augaugaugau, augau, auaaaagaua, or auaaaagaug. In some embodiments, the RNA molecule may include (i) an ORF encoding a replicase which may transcribe RNA from a second RNA molecule and (ii) an ORF encoding at least one antigen or polypeptide of interest. In some embodiments, an open reading frame of an RNA (e.g., saRNA) composition is codon-optimized.

### Influenza Vaccines

Some embodiments provide influenza vaccines comprising one or more RNA polynucleotides having an open reading frame encoding a hemagglutinin (HA) protein, or a fragment thereof, and a pharmaceutically acceptable carrier or excipient, formulated within a cationic lipid nanoparticle. In some embodiments, the hemagglutinin protein is selected from H1, H7 and H10. In some embodiments, the RNA polynucleotide further encodes neuraminidase (NA) protein. In some embodiments, the Influenza virus is selected from H1N1, H3N2, H7N9, and H10N8. In some embodiments, the hemagglutinin protein is H1, H2, H3, H4, H5, H6, H7, H8, H9, H10, H11, H12, H13, H14, H15, H16, H17, H18, or an immunogenic fragment thereof. In some embodiments, the hemagglutinin protein does not comprise a head domain. In some embodiments, the hemagglutinin protein comprises a portion of the head domain. In some embodiments, the hemagglutinin protein does not comprise a cytoplasmic domain. In some embodiments, the hemagglutinin protein comprises a portion of the cytoplasmic domain. In some embodiments, the truncated hemagglutinin protein comprises a portion of the transmembrane domain.

In some embodiments, the virus is a strain of Influenza A or Influenza B or combinations thereof. In some embodiments, the strain of Influenza A or Influenza B is associated with birds, pigs, horses, dogs, humans, or non-human primates. Protein fragments, functional protein domains, and homologous proteins are also considered to be within the scope of polypeptides of interest. For example, provided herein is any protein fragment (meaning a polypeptide sequence at least one amino acid residue shorter than a reference polypeptide sequence but otherwise identical) of a reference protein 10, 20, 30, 40, 50, 60, 70, 80, 90, 100 or greater than 100 amino acids in length.

In some embodiments, the at least one antigenic influenza polypeptide is one of the defined antigenic subdomains of HA, termed HA1, HA2, or a combination of HA1 and HA2, and at least one antigenic polypeptide selected from neuraminidase (NA), nucleoprotein (NP), matrix protein 1 (M1), matrix protein 2 (M2), non-structural protein 1 (NS1) and non-structural protein 2 (NS2). In some embodiments, the at least one antigenic polypeptide is HA or derivatives thereof comprising antigenic sequences from HA1 and/or HA2, and at least one antigenic polypeptide selected from NA, NP, M1, M2, NS1 and NS2.

In some embodiments, an influenza RNA composition includes an RNA encoding an antigenic fusion protein. Thus, the encoded antigen or antigens may include two or more proteins (e.g., protein and/or protein fragment) joined together. Alternatively, the protein to which a protein antigen is fused does not promote a strong immune response to itself, but rather to the influenza antigen. Antigenic fusion proteins, in some embodiments, retain the functional property from each original protein.

### 5′ Cap

In some embodiments, the saRNA molecule described herein includes a 5′ cap. In some embodiments, the 5′-cap moiety is a natural 5-cap. A “natural 5′-cap” is defined as a cap that includes 7-methylguanosine connected to the 5′ end of an mRNA molecule through a 5′ to 5′ triphosphate linkage. In some embodiments, the 5′-cap moiety is a 5′-cap analog. In some embodiments, the 5′ end of the RNA is capped with a modified ribonucleotide with the structure m7G (5′) ppp (5′) N (cap 0 structure) or a derivative thereof, which may be incorporated during RNA synthesis (e.g., co-transcriptional capping) or may be enzymatically engineered after RNA transcription (e.g., post-transcriptional capping), wherein “N” is any ribonucleotide. In some embodiments, the 5′ end of the RNA molecule is capped with a modified ribonucleotide via an enzymatic reaction after RNA transcription. In some embodiments, capping is performed after purification, e.g., tangential flow filtration, of the RNA molecule. An exemplary enzymatic reaction for capping may include use of Vaccinia Virus Capping Enzyme (VCE) that includes mRNA triphosphatase, guanylyl-transferase, and guanine-7-methytransferase, which catalyzes the construction of N7-monomethylated cap 0 structures. Cap 0 structure can help maintaining the stability and translational efficacy of the RNA molecule. The 5′ cap of the RNA molecule may be further modified by a 2′-O-methyltransferase which results in the generation of a cap 1 structure (m7Gppp [m2′-O]N), which may further increase translation efficacy. In some embodiments, the RNA molecule may be enzymatically capped at the 5′ end using Vaccinia guanylyltransferase, guanosine triphosphate, and S-adenosyl-L-methionine to yield cap 0 structure. An inverted 7-methylguanosine cap is added via a 5′ to 5′ triphosphate bridge. Alternatively, use of a 2′O-methyltransferase with Vaccinia guanylyltransferase yields the cap 1 structure where in addition to the cap 0 structure, the 2′OH group is methylated on the penultimate nucleotide. S-adenosyl-L-methionine (SAM) is a cofactor utilized as a methyl transfer reagent.

Non-limiting examples of 5′ cap structures are those which, among other things, have enhanced binding of cap binding polypeptides, increased half-life, reduced susceptibility to 5′ endonucleases and/or reduced 5′ de-capping, as compared to synthetic 5′ cap structures known in the art (or to a wild-type, natural or physiological 5′ cap structure). For example, recombinant Vaccinia Virus Capping Enzyme and recombinant 2′-O-methyltransferase enzyme may create a canonical 5′-5′-triphosphate linkage between the 5′-terminal nucleotide of an mRNA and a guanine cap nucleotide wherein the cap guanine includes an N7 methylation and the 5′-terminal nucleotide of the mRNA includes a 2′-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N,pN2p (cap 0) and 7mG(5′)ppp(5′)N1mpNp (cap 1). Cap 0 is a N7-methyl guanosine connected to the 5′ nucleotide through a 5′ to 5′ triphosphate linkage, typically referred to as m7G cap or m7Gppp. In the cell, the cap 0 structure can help provide for efficient translation of the mRNA that carries the cap. An additional methylation on the 2′O position of the initiating nucleotide generates Cap 1, or refers to as m7GpppNm-, wherein Nm denotes any nucleotide with a 2′O methylation. In some embodiments, the 5′terminal cap includes a cap analog, for example, a 5′ terminal cap may include a guanine analog. Exemplary guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine. In some embodiments, the capping region may include a single cap or a series of nucleotides forming the cap. In this embodiment the capping region may be equal to any one of, at least any one of, at most any one of, or between any two of 1, 2, 3, 4, 5, 6, 7, 8, 9, 10, or at least 2, or 10 or fewer nucleotides in length. In some embodiments, the cap is absent. In some embodiments, the first and second operational regions may be equal to any one of, at least any one of, at most any one of, or between any two of 3 to 40, e.g., 5 to 30, 10 to 20, 15, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or at least 4, or 30 or fewer nucleotides in length and may comprise, in addition to a start and/or stop codon, one or more signal and/or restriction sequences. In some embodiments, the 5′ cap comprises CLEANCAP® Reagent AG (3′ OMe) for co-transcriptional capping of mRNA. In some embodiments, the 5′cap comprises CLEANCAP® AU for self-amplifying mRNA, or CLEANCAP® Reagent AU for co-transcriptional capping of mRNA.

### Poly-A Tail

As used herein, “poly A tail” refers to a stretch of consecutive adenine residues, which may be attached to the 3′ end of the RNA molecule. The poly-A tail may increase the half-life of the RNA molecule. Poly-A tails may play key regulatory roles in enhancing translation efficiency and regulating the efficiency of mRNA quality control and degradation. Short sequences or hyperpolyadenylation may signal for RNA degradation. Exemplary designs include a poly-A tails of about 40 adenine residues to about 80 adenine residues. In some embodiments, the RNA molecule further includes an endonuclease recognition site sequence immediately downstream of the poly A tail sequence. In some embodiments, the RNA molecule further includes a poly-A polymerase recognition sequence (e.g., AAUAAA) near its 3′ end. A “full length” RNA molecule is one that includes a 5′-cap and a poly A tail.

In some embodiments, the poly A tail includes 5 to 400 nucleotides in length. The poly A tail nucleotide length may be equal to any one of, at least any one of, at most any one of, or between any two of 5, 6, 7, 8, 9, 10, 15, 20, 25. 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, and 400. In some embodiments, the RNA molecule includes a poly A tail that includes about 25 to about 400 adenosine nucleotides, a sequence of about 50 to about 400 adenosine nucleotides, a sequence of about 50 to about 300 adenosine nucleotides, a sequence of about 50 to about 250 adenosine nucleotides, a sequence of about 60 to about 250 adenosine nucleotides, or a sequence of about 40 to about 100 adenosine nucleotides.

### Lipid Delivery

Lipids (e.g. lipid nanoparticles—“LNPs”) are known to those of skill in the art as useful for the in vivo delivery of nucleic acids, such as RNA. As used herein, the term “lipid nanoparticle”, or “LNP”, refers to particles of any morphology generated when a cationic lipid and optionally one or more further lipids are combined, e.g. in an aqueous environment and/or in the presence of a nucleic acid such as RNA. In some aspects, lipid nanoparticles are included in a formulation that can be used to deliver an active agent or therapeutic agent, such as a nucleic acid (e.g., mRNA, saRNA) to a target site of interest (e.g., cell, tissue, organ, tumor, and the like). In some aspects, lipid nanoparticles of the present disclosure comprise a nucleic acid. Such lipid nanoparticles typically comprise a cationic lipid and one or more excipients, e.g., one or more neutral lipids, charged lipids, steroids, polymer conjugated lipids, or combinations thereof. In some aspects, the active agent or therapeutic agent, such as a nucleic acid (e.g., mRNA, saRNA), may be encapsulated in the lipid portion of the lipid nanoparticle or an aqueous space enveloped by some or all of the lipid portion of the LNP, thereby protecting it from enzymatic degradation or other undesirable effects induced by the mechanisms of the host organism or cells e.g. an adverse immune response. The nucleic acid (e.g., mRNA, saRNA) or a portion thereof may also be associated and complexed with the LNP. A lipid nanoparticle may comprise any lipid capable of forming a particle to which the nucleic acids are attached, or in which the one or more nucleic acids are encapsulated. The LNPs disclosed herein typically have a mean diameter of from about 1 nm to about 500 nm. The term “mean diameter” refers to the mean hydrodynamic diameter of particles as measured by dynamic laser light scattering (DLS) with data analysis using the so-called cumulant algorithm, which provides as results the so-called Z-average with the dimension of a length, and the polydispersity index (PI), which is dimensionless (Koppel, D., J. Chem. Phys. 57, 1972, pp 4814-4820, ISO 13321). Here, “mean diameter,” “diameter,” or “size” for particles is used synonymously with this value of the Z-average.

The term “polymer-lipid conjugate” refers to a molecule comprising both a lipid portion and a polymer portion. An example of a polymer-lipid conjugate is a pegylated lipid. The term “pegylated lipid” (PEG lipid) refers to a molecule comprising both a lipid portion and a polyethylene glycol portion. Pegylated lipids are known in the art and include lipids such as 1-(monomethoxy-polyethyleneglycol)-2,3-dimyristoylglycerol (PEG-s-DMG), 2-[(polyethylene glycol)-2000]-N,N-ditetradecylacetamide, poly-ethylene glycol conjugated lipids in the C14 to C18 range, i.e. DMPC-PEG (Dimyristoylphosphatidylcholine-polyethylene glycol-2000), DMG-PEG (1,2-dimyristoyl-rac-glycero-3-methoxypolyethylene glycol-2000), DOPE-PEG (1,2-dioleoyl-sn-glycero-3-phosphoethanolamine-N-[amino(polyethylene glycol)-2000]ammonium salt), DSPE-PEG (1,2-Distearoyl-sn-glycero-3-phosphoethanolamine-Poly (ethylene glycol)-2000), ALC-0159 (2-[(polyethylene glycol)-2000]-N,N-ditetradecylacetamide), oleoyl polyethylene glycol 2000, and poly(ethylene glycol)-block-poly(propylene glycol)-block-poly(ethylene glycol), and the like. Other examples of polymer-lipid conjugates include polyoxazolines, poly (2-methyl-2-oxazoline) (PMOZ), poly (2-ethyl-2-oxazoline) (PEOZ), polysarcosines, polyglycerol polymer conjugates (i.e. PG-30), and polycarbonate polymer conjugates (i.e. PCs) and the like.

As used herein, a “cationic lipid” refers to a lipid or lipid like material having a net positive charge. Cationic lipids bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge. Exemplary cationic lipids include one or more amine group(s) which bear the positive charge. Cationic lipids may encapsulate negatively charged RNA. In some aspects, cationic lipids are ionizable such that they can exist in a positively charged or neutral form depending on pH. The ionization of the cationic lipid affects the surface charge of the lipid nanoparticle under different pH conditions. Without wishing to be bound by theory, this ionizable behavior is thought to enhance efficacy through helping with endosomal escape and reducing toxicity as compared with particles that remain cationic at physiological pH. For purposes of the present disclosure, such “cationically ionizable” lipids are comprised by the term “cationic lipid” unless contradicted by the circumstances. Without limitation, examples of cationic lipids include Dlin-MC3-DMA ((6Z,9Z,28Z,31Z)-Heptatriaconta-6,9,28,31-tetraen-19-yl 4-(dimethylamino)butanoate), ALC-0315 ([(4-hydroxybutyl)azanediyl]di(hexane-6,1-diyl) bis(2-hexyldecanoate)), DLin-KC2-DMA (2-[2,2-bis[(9Z,12Z)-octadeca-9,12-dienyl]-1,3-dioxolan-4-yl]-N,N-dimethylethanamine), DLinDMA (1,2-dilinoleyloxy-n,n-dimethyl-3-aminopropane), DODMA (N,N-dimethyl-2,3-bis[(Z)-octadec-9-enoxy]propan-1-amine), DOGS (5-carboxyspermylglycinedioctadecylamide), DODAP (1,2-Dioleoyl-3-Dimethylammonium-Propane), DOSPA (2,3-dioleyloxy-N-[2(spermine-carboxamido)ethyl]-N,N-dimethyl-1-propanaminium), DOTAP (1,2-Dioleoyl-3-Trimethylammonium-Propane), DSDMA (1,2-distearyloxy-N,N-dimethyl-3-aminopropane), DLenDMA (1,2-dilinolenyloxy-N,N-dimethyl-3-aminopropane), DODAC (N-dioleyl-N,N-dimethylammonium chloride), DDAB (N,N-distearyl-N,N-dimethylammonium bromide), DMRIE (N-[1,2-dimyristyloxyprop-3-yl]-N,N-dimethyl-N-hydroxyethyl ammonium bromide), CLinDMA (3-dimethylamino-2-(cholest-5-en-3-beta-oxybutan-4-oxy)-1-(cis,cis-9,12-oc-tadecadienoxy)propane), CpLinDMA (2-[5′-(cholest-5-en-3-beta-oxy)-3′-oxapentoxy)-3-dimethy 1-1-(cis,cis-9′,1-2′-octadecadienoxy)propane), and DMOBA (N,N-dimethyl-3,4-dioleyloxybenzylamine).

As used herein, a “structural lipid” is a lipid that helps stabilize the formation of LNPs during their formation, and can mediate the morphology of an LNP. Such structural lipids (sometimes referred to as “helper lipids”) include non-cationic lipids, e.g., neutral lipids and anionic lipids (e.g. phospholipids). Without limitation, examples of structural lipids include monoacyglycerols, aliphatic alcohols, and sterols. Without wishing to be bound by any theory, optimizing the formulation of LNPs by addition of such structural lipids in addition to a cationic lipid, may enhance particle stability and efficacy of nucleic acid delivery.

As used herein, the term “phospholipid” is any lipid that comprises a phosphate group. Phospholipids are a subset of non-cationic lipids. Without limitation, examples of phospholipids include 1,2-Dioleoyl-phosphatidic acid (DOPA), 1,2-Dioleoyl-phosphatidylglycerol (DOPG), 1,2-Distearoyl-phophatidylglycerol (DSPG), 1,2-Dioleoyl-phosphatidylethanolamine (DOPE), 1,2-distearoyl-glycero-3-phosphoethanolamine (DSPE), 1,2-Dioleoyl-phosphatidylcholine (DOPC), 1-Palmitoyl-2-oleoyl-sn-glycero-3-phosphocholine (POPC), 1,2-Dioleoyl-sn-glycero-3-phosphoserine (DOPS), 1,2-Dipalmitoylphosphatidylserine (DPPS), lyso1-hydroxy-2-oleoyl-sn-glycero-3 -phosphocholine, 1,2-dioleoyl-sn-glycero-3-dihexyl-phosphocholine, and distearoylphosphatidylcholine (DSPC).

As used herein, sterols are a subgroup of steroids consisting of steroid alcohols, and are known to those in the art. Without limitation, examples of sterols include cholesterol, fecosterol, sitosterol, ergosterol, campesterol, stigmasterol, brassicasterol, and derivatives thereof such as cholestanol, tocopherol, cholestanone, cholestenone, coprostanol, cholesteryl-2′-hydroxyethyl ether, cholesteryl-4′-hydroxybutyl ether, and mixtures thereof.

Without intending to be bound by any theory, it is believed that the cationic or cationically ionizable lipid or lipid-like material and/or the cationic polymer combine together with the nucleic acid to form aggregates, and this aggregation results in colloidally stable particles. A lipid may be a naturally occurring lipid or a synthetic lipid. However, a lipid is usually a biological substance. Biological lipids are well known in the art, and include for example, neutral fats, phospholipids, phosphoglycerides, steroids, terpenes, lysolipids, glycosphingolipids, glucolipids, sulphatides, lipids with ether and ester-linked fatty acids and polymerizable lipids, and combinations thereof. A lipid is a substance that is insoluble in water and extractable with an organic solvent. Compounds other than those specifically described herein are understood by one of skill in the art as lipids, and are encompassed by the compositions and methods of the present disclosure. A lipid component and a non-lipid may be attached to one another, either covalently or non-covalently.

In some aspects, LNPs can be designed to protect RNAs (e.g., saRNA, mRNA) from extracellular RNases and/or can be engineered for systemic delivery of the RNA to target cells. In some aspects, such LNPs may be particularly useful to deliver RNAs when RNAs are intravenously administered to a subject in need thereof. In some aspects, such LNPs may be particularly useful to deliver RNAs when RNAs are intramuscularly administered to a subject in need thereof. Accordingly, the LNPs described herein can further be administered via intravenous, intramuscular, subcutaneous, intratumoral, intranasal, or inhalation routes of administration.

In some aspects, the lipid nanoparticles may have an average particle size (as measured by their mean diameter) of about 1 to 500 nm, or about 20 to 500 nm. In some aspects, the lipid nanoparticles have a mean diameter of from about 20 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or at least, at most, exactly, or between any two of 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. The term “mean diameter” refers to the mean hydrodynamic diameter of particles as measured by dynamic laser light scattering (DLS) with data analysis using the so-called cumulant algorithm, which provides as results the so-called Z-average with the dimension of a length, and the polydispersity index (PI), which is dimensionless (Koppel, D., J. Chem. Phys. 57, 1972, pp 4814-4820, ISO 13321).

LNPs described herein may exhibit a polydispersity index less than about 0.5, less than about 0.4, less than about 0.3, or about 0.2 or less. By way of example, the LNPs can exhibit a polydispersity index of at least, at most, exactly, or between any two of 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5. The polydispersity index is, in some aspects, calculated based on dynamic light scattering measurements by the so-called cumulant analysis as mentioned in the definition of the “average diameter.” Under certain prerequisites, it can be taken as a measure of the size distribution of an ensemble of nanoparticles.

In certain aspects, nucleic acids (e.g., RNAs), when present in LNPs, are resistant in aqueous solution to degradation with a nuclease. In some aspects, LNPs are liver-targeting lipid nanoparticles.

In certain aspects, the RNA solution and lipid preparation mixture or compositions thereof may have, have at least, have at most, have exactly, or have between any two of about 1%, about 2%, about 3%, about 4% about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% of a particular lipid, lipid type, or non-lipid component such as lipid-like materials and/or cationic polymers or an adjuvant, antigen, peptide, polypeptide, sugar, nucleic acid or other material disclosed herein or as would be known to one of skill in the art.

LNPs described herein can be prepared using a wide range of methods that may involve obtaining a colloid from at least one cationic or cationically ionizable lipid or lipid-like material and/or at least one cationic polymer and mixing the colloid with nucleic acid to obtain nucleic acid particles. The term “colloid” as used herein relates to a type of homogeneous mixture in which dispersed particles do not settle out. The insoluble particles in the mixture are microscopic, with particle sizes between 1 and 1000 nm. The mixture may be termed a colloid or a colloidal suspension. Sometimes the term “colloid” only refers to the particles in the mixture and not the entire suspension.

For the preparation of colloids comprising at least one cationic or cationically ionizable lipid or lipid-like material and/or at least one cationic polymer, methods are applicable herein that are conventionally used for preparing liposomal vesicles and are appropriately adapted. The most commonly used methods for preparing liposomal vesicles share the following fundamental stages: (i) lipids dissolution in organic solvents, (ii) drying of the resultant solution, and (iii) hydration of dried lipid (using various aqueous media). In the film hydration method, lipids are firstly dissolved in a suitable organic solvent, and dried down to yield a thin film at the bottom of the flask. The obtained lipid film is hydrated using an appropriate aqueous medium to produce a liposomal dispersion. Furthermore, an additional downsizing step may be included.

Reverse phase evaporation is an alternative method to the film hydration for preparing liposomal vesicles that involves formation of a water-in-oil emulsion between an aqueous phase and an organic phase containing lipids. A brief sonication of this mixture is required for system homogenization. The removal of the organic phase under reduced pressure yields a milky gel that turns subsequently into a liposomal suspension.

The term “ethanol injection technique” refers to a process, in which an ethanol solution comprising lipids is rapidly injected into an aqueous solution through a needle. This action disperses the lipids throughout the solution and promotes lipid structure formation, for example lipid vesicle formation such as liposome formation. Generally, the RNA lipoplex particles described herein are obtainable by adding RNA to a colloidal liposome dispersion. Using the ethanol injection technique, such colloidal liposome dispersion is, in some embodiments, formed as follows: an ethanol solution comprising lipids, such as cationic lipids and additional lipids, is injected into an aqueous solution under stirring. In some embodiments, the RNA lipoplex particles described herein are obtainable without a step of extrusion.

The term “extruding” or “extrusion” refers to the creation of particles having a fixed, cross-sectional profile. In particular, it refers to the downsizing of a particle, whereby the particle is forced through filters with defined pores.

Other methods having organic solvent free characteristics may also be used according to the present disclosure for preparing a colloid.

In some aspects, LNP-encapsulated RNA can be produced by rapid mixing of an RNA solution described herein (e.g., the RNA product solution) and a lipid preparation described herein (comprising, e.g., at least one cationic lipid and optionally one or more other lipid components, in an organic solvent) under conditions such that a sudden change in solubility of lipid component(s) is triggered, which drives the lipids towards self-assembly in the form of LNPs. In some aspects, suitable buffering agents comprise tris, histidine, citrate, acetate, phosphate, or succinate. The pH of a liquid formulation relates to the pKa of the encapsulating agent (e.g. cationic lipid). The pH of the acidifying buffer may be at least half a pH scale less than the pKa of the encapsulating agent (e.g. cationic lipid), and the pH of the final buffer may be at least half a pH scale greater than the pKa of the encapsulating agent (e.g. cationic lipid). In some aspects, properties of a cationic lipid are chosen such that nascent formation of particles occurs by association with an oppositely charged backbone of a nucleic acid (e.g., RNA). In this way, particles are formed around the nucleic acid, which, for example, in some aspects, can result in much higher encapsulation efficiency than is achieved in the absence of interactions between nucleic acids and at least one of the lipid components.

In certain aspects, nucleic acids, when present in the lipid nanoparticles, are resistant in aqueous solution to degradation with a nuclease. Lipid nanoparticles comprising nucleic acids and their method of preparation are disclosed in, e.g., U.S. Patent Publication Nos. 2004/0142025, 2007/0042031 and PCT Pub. Nos. WO 2013/016058 and WO 2013/086373, the full disclosures of which are herein incorporated by reference in their entirety for all purposes.

Some embodiments described herein relate to compositions, methods and uses involving more than one, e.g., 2, 3, 4, 5, 6 or even more nucleic acid species such as RNA species. In an LNP formulation, it is possible that each nucleic acid species is separately formulated as an individual LNP formulation. In that case, each individual LNP formulation will comprise one nucleic acid species. The individual LNP formulations may be present as separate entities, e.g. in separate containers. Such formulations are obtainable by providing each nucleic acid species separately (typically each in the form of a nucleic acid-containing solution) together with suitable cationic or cationically ionizable lipids or lipid-like materials and cationic polymers that allow the formation of LNPs. Respective particles will contain exclusively the specific nucleic acid species that is being provided when the particles are formed (individual particulate formulations).

In some embodiments, a composition such as a pharmaceutical composition comprises more than one individual LNP formulation. Respective pharmaceutical compositions can be referred to as mixed LNP formulations. Mixed LNP formulations according to the disclosure can be obtained by forming, separately, individual LNP formulations, as described above, followed by a step of mixing of the individual LNP formulations. By the step of mixing, a formulation comprising a mixed population of nucleic acid-containing LNPs is obtainable. Individual LNP populations may be together in one container, comprising a mixed population of individual LNP formulations.

Alternatively, it is possible that different nucleic acid species are formulated together as a combined LNP formulation. Such formulations are obtainable by providing a combined formulation (typically combined solution) of different RNA species together with suitable cationic or cationically ionizable lipids or lipid-like materials and cationic polymers that allow the formation of LNPs. As opposed to a mixed LNP formulation, a combined LNP formulation will typically comprise LNPs that comprise more than one RNA species. In a combined LNP composition, different RNA species are typically present together in a single particle.

In one aspect, the present disclosure provides compounds that modulate the innate immune response that can be included within the LNP formulation. Alternatively, as discussed herein, such compounds can be administered in a formulation that is separate from the LNP formulation. In addition to the components and compounds discussed herein, lipid nanoparticles may include any substance useful in pharmaceutical compositions. For example, the lipid nanoparticle may include one or more pharmaceutically acceptable excipients or accessory ingredients such as, but not limited to, one or more solvents, dispersion media, diluents, dispersion aids, suspension aids, surface active agents, buffering agents, preservatives, and other species.

In some embodiments, the formulation including a LNP may further include a salt, such as a chloride salt. In some embodiments, the formulation including a LNP may further include a sugar such as a disaccharide. In some embodiments, the formulation further includes a sugar but not a salt, such as a chloride salt. In some embodiments, a LNP may further include one or more small hydrophobic molecules such as a vitamin (e.g., vitamin A or vitamin E) or a sterol. Carbohydrates may include simple sugars (e.g., glucose) and polysaccharides (e.g., glycogen and derivatives and analogs thereof).

Pharmaceutical compositions that include an LNP may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21st Edition, A. R. Gennaro; Lippincott, Williams & Wilkins, Baltimore, MD, 2006. Conventional excipients and accessory ingredients may be used in any pharmaceutical composition, except insofar as any conventional excipient or accessory ingredient may be incompatible with one or more components of a LNP or the one or more amphiphilic polymers in the formulation of the disclosure. An excipient or accessory ingredient may be incompatible with a component of a LNP or the amphiphilic polymer of the formulation if its combination with the component or amphiphilic polymer may result in any undesirable biological effect or otherwise deleterious effect.

In some embodiments, one or more excipients or accessory ingredients may make up greater than 50% of the total mass or volume of a pharmaceutical composition including a LNP. For example, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia. Relative amounts of the one or more amphiphilic polymers, the one or more lipid nanoparticles, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition may comprise between 0.1% and 100% (wt/wt) of one or more lipid nanoparticles. As another example, a pharmaceutical composition may comprise between 0.1% and 15% (wt/vol) of one or more amphiphilic polymers (e.g., 0.5%, 1%, 2.5%, 5%, 10%, or 12.5% w/v).

An LNP may optionally comprise one or more coatings. For example, a LNP may be formulated in a capsule, film, or tablet having a coating. A capsule, film, or tablet including a composition described herein may have any useful size, tensile strength, hardness, or density.

In certain embodiments, the lipid nanoparticles and/or pharmaceutical compositions of the disclosure are refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). For example, the pharmaceutical composition comprising one or more amphiphilic polymers and one or more lipid nanoparticles is a solution or solid (e.g., via lyophilization) that is refrigerated for storage and/or shipment at, for example, about −20° C., −30° C., −40° C., −50° C., −60° C., −70° C., or −80° C. In certain embodiments, the disclosure also relates to a method of increasing stability of the lipid nanoparticles by adding an effective amount of an amphiphilic polymer and by storing the lipid nanoparticles and/or pharmaceutical compositions thereof at a temperature of 4° C. or lower, such as a temperature between about −150° C. and about 0° C. or between about −80° C. and about −20° C., e.g., about −5° C., −10° C., −15° C., −20° C., −25° C., −30° C., −40° C., −50° C., −60° C., −70° C., −80° C., −90° C., −130° C. or −150° C.).

### Compositions

In some embodiments, the present disclosure provides a composition that comprises at least one nucleic acid (such as saRNA) that is co-formulated with at least one compound that modulates the innate immune response to the nucleic acid when administered to a subject in need thereof. For example, in some embodiments the nucleic acid is RNA, such as saRNA. In some embodiments, at least 10%, at least 20%, at least 30%, at least 40%, at least 50%, at least 60%, at least 70%, at least 80%, or at least 90% of the total nucleotides in the RNA have been replaced with modified or unnatural nucleotides.

The saRNAs of the present disclosure can have multiple reading frames that encode various genes of interest. For example, in some embodiments such genes of interest include those derived from influenza virus antigens, such as hemagglutinin (HA) and or neuraminidase (NA). Such genes of interest can be derived from any strain of the influenza virus.

The compositions of the present disclosure can comprise a nucleic acid and a compound that modulates the innate immune response encapsulated in a lipid nanoparticle (LNP), for example an LNP that comprises a cationic lipid. Such LNPs can further comprise a phospholipid, a steroid, and/or a PEG lipid.

In some embodiments, the compounds of the present disclosure that modulate the innate immune response to a nucleic acid are modulators of IRAK4, such as 1-{[(2S,3S,4S)-3-ethyl-4-fluoro-5-oxopyrrolidin-2-yl]methoxy}-7-methoxyisoquinoline-6-carboxamide, 1-{[(2S,3S,4S)-4-fluoro-3-methyl-5-oxopyrrolidin-2-yl]methoxy}-7-methoxyisoquinoline-6-carboxamide, N-{3-(difluoromethyl)-1-[(1S,4R)-4-({4-[(3-{1-[(3S)-2,6-dioxopiperidin-3-yl]-3-methyl-2-oxo-2,3-dihydro-1H-benzimidazol-4-yl}prop-2-yn-1-yl)oxy]piperidin-1-yl}methyl)cyclohexyl]-1H-pyrazol-4-yl}-5-[(1R,4R)-2-oxa-5-azabicyclo[2,2,1]heptan-5-yl]pyrazolo[1,5-a]pyrimidine-3-carboxamide, and N-{2-[(1R,4r)-4-({[1-({1-[(3R)-2,6-dioxopiperidin-3-yl]-3-methyl-2-oxo-2,3-dihydro-1H-benzimidazol-4-yl}methyl)piperidin-4-yl](methyl)amino}methyl)cyclohexyl]-6-(2-hydroxypropan-2-yl)-2H-indazol-5-yl}-6-(trifluoromethyl)pyridine-2-carboxamide, or pharmaceutically acceptable salts thereof. In further embodiments, such compounds are modulators of RIG-I, such as 2,2′-(piperazine-1,4-diyl)bis(6,7-dimethoxyquinazolin-4-amine) and 6,7-dimethoxy-2-[4-(2-methylpyrimidin-4-yl)piperazin-1-yl]quinazolin-4-amine, or pharmaceutically acceptable salts thereof. Such compounds can each be present in an effective amount. Compositions suitable for use in the present disclosure include compositions wherein the compound that modulates the innate immune response is present in an amount sufficient to achieve the intended purpose, i.e., to effectively modulate the innate immune response.

### Methods of Use

The compositions of the present disclosure may be useful for treating or preventing a disease, disorder, or condition or delivering an active agent to treat or prevent a disease, disorder, or condition. In particular, such compositions may be useful in treating or preventing a disease, disorder, or condition characterized by missing or aberrant protein or polypeptide activity. Diseases, disorders, and/or conditions characterized by dysfunctional or aberrant protein or polypeptide activity for which a composition may be administered include, but are not limited to rare diseases, infectious diseases (as both vaccines and therapeutics), cancer and proliferative diseases, genetic diseases, autoimmune diseases, diabetes, neurodegenerative diseases, cardio- and reno-vascular diseases, and metabolic diseases. Such compositions may also be useful to elicit an immune response against a particular antigen.

In some embodiments, the present disclosure provides methods of using IRAK4 and RIG-1 compounds to modulate the innate immune response to a nucleic acid, such as the nucleic acids disclosed herein, including saRNA, when administered to a subject in need thereof. Such methods include use of any of the compositions as disclosed herein. To achieve modulation of the innate immune response to a nucleic acid, such IRAK4 and/or RIG-1 compounds can be administered in an effective amount to the subject as a co-formulation such that the compound and the nucleic acid are present in the same formulation. Alternatively, the nucleic acid and the compound can be administered as effective amounts as separate formulations, but are administered simultaneously. Even further, the compound and the nucleic acid can be administered in effective amounts before or after the other. For example, the nucleic acid can be administered in an effective amount first, followed by the administration of the compound in an effective amount; or the compound can be administered first, followed by the administration of the nucleic acid. The time between such administrations can be one or up to several hours, such as 1 to 24 hours. Even further, the time between such administrations can be one, or up to several days or weeks, or months.

In some embodiments, the nucleic acid is RNA, such as saRNA. In some embodiments, at least 10%, at least 20%, at least 30%, at least 40%, at least 50%, at least 60%, at least 70%, at least 80%, or at least 90% of the total nucleotides in the RNA have been replaced with modified or unnatural nucleotides.

In some embodiments, said effective amount is about 0.01 to about 100 mg/kg of body weight/day, or more preferably about 0.1 to about 10.0 mg/kg, in a single dose or as divided doses administered two, three or four times per day. The disclosure also provides the method, wherein said therapeutically effective amount is about 400 mg administered QD. The disclosure further provides the method, wherein said therapeutically effective amount is about 200 mg administered Q6H.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations such as multiple inhalations from an insufflator or by application of a plurality of drops into the eye.

Any of the compounds disclosed herein, or their pharmaceutically acceptable salts, can be administered orally, parenterally, topically, rectally, transmucosally, or intestinally. Parenteral administrations include indirect injections to generate a systemic effect or direct injections to the afflicted area. Topical administrations include the treatment of skin or organs readily accessible by local application, for example, eyes or ears. It also includes transdermal delivery to generate a systemic effect. The rectal administration includes the form of suppositories.

Some aspects of the disclosure are directed to a method of inducing an immune response in a subject, comprising administering to the subject in need thereof an effective amount of a composition as disclosed herein. Some aspects of the disclosure are directed to a method of vaccinating a subject, comprising administering to the subject in need thereof an effective amount of a composition as disclosed herein. Some aspects of the disclosure are directed to a method comprising administering to the subject in need thereof an effective amount of a composition as disclosed herein. In some embodiments, a composition as disclosed herein elicits an immune response comprising an antibody response. In some embodiments, a composition as disclosed herein elicits an immune response comprising a T cell response.

Some embodiments of the present disclosure provide methods of inducing an antigen specific immune response in a subject, comprising administering to the subject any of the RNA (e.g., saRNA) compositions as provided herein in an amount effective to produce an antigen-specific immune response. In some embodiments, the RNA (e.g., saRNA) composition is an influenza vaccine. In some embodiments, the RNA (e.g., saRNA) composition is a combination vaccine comprising a combination of influenza vaccines (a broad spectrum influenza vaccine).

In some embodiments, an antigen-specific immune response comprises a T cell response or a B cell response. In some embodiments, a method of producing an antigen-specific immune response comprises administering to a subject a single dose (no booster dose) of an influenza RNA (e.g., saRNA) composition of the present disclosure. In some embodiments, a method further comprises administering to the subject a second (booster) dose of an influenza RNA (e.g., saRNA) composition. Additional doses of an influenza RNA (e.g., saRNA) composition may be administered.

In some embodiments, the subjects exhibit a seroconversion rate of at least 80% (e.g., at least 85%, at least 90%, or at least 95%) following the first dose or the second (booster) dose of the vaccine. Seroconversion is the time period during which a specific antibody develops and becomes detectable in the blood. After seroconversion has occurred, a virus can be detected in blood tests for the antibody. During an infection or immunization, antigens enter the blood, and the immune system begins to produce antibodies in response. Before seroconversion, the antigen itself may or may not be detectable, but antibodies are considered absent. During seroconversion, antibodies are present but not yet detectable. Any time after seroconversion, the antibodies can be detected in the blood, indicating a prior or current infection.

In some embodiments, an influenza RNA (e.g., saRNA) composition is administered to a subject by intradermal injection, intramuscular injection, or by intranasal administration. In some embodiments, an influenza RNA (e.g., saRNA) composition is administered to a subject by intramuscular injection.

Some embodiments, of the present disclosure provide methods of inducing an antigen specific immune response in a subject, including administering to a subject an influenza RNA (e.g., saRNA) composition in an effective amount to produce an antigen specific immune response in a subject. Antigen-specific immune responses in a subject may be determined, in some embodiments, by assaying for antibody titer (for titer of an antibody that binds to an influenza antigenic polypeptide) following administration to the subject of any of the influenza RNA (e.g., saRNA) compositions of the present disclosure. In some embodiments, the anti-antigenic polypeptide antibody titer produced in the subject is increased by at least 1 log relative to a control. In some embodiments, the anti-antigenic polypeptide antibody titer produced in the subject is increased by 1 to 3 log relative to a control.

In some embodiments, the anti-antigenic polypeptide antibody titer produced in a subject is increased at least 2 times relative to a control. In some embodiments, the anti-antigenic polypeptide antibody titer produced in the subject is increased at least 5 times relative to a control. In some embodiments, the anti-antigenic polypeptide antibody titer produced in the subject is increased at least 10 times relative to a control. In some embodiments, the anti-antigenic polypeptide antibody titer produced in the subject is increased 2 to 10 times relative to a control.

In some embodiments, the control is an anti-antigenic polypeptide antibody titer produced in a subject who has not been administered a RNA (e.g., saRNA) composition of the present disclosure. In some embodiments, the control is an anti-antigenic polypeptide antibody titer produced in a subject who has been administered a live attenuated or inactivated influenza, or wherein the control is an anti-antigenic polypeptide antibody titer produced in a subject who has been administered a recombinant or purified influenza protein vaccine.

In some embodiments, the RNA (e.g., saRNA) composition is formulated in an effective amount to produce an antigen specific immune response in a subject.

In some embodiments, the effective amount is a total dose of 1 μg to 1000 μg, or 1 μg to 100 μg of saRNA. In some embodiments, the effective amount is a total dose of 30 μg. In some embodiments, the effective amount is a dose of 10 μg administered to the subject a total of two times. In some embodiments, the effective amount is a dose of 10 μg administered to the subject a total of two times. In some embodiments, the effective amount is a dose of 15 μg administered to the subject a total of two times. In some embodiments, the effective amount is a dose of 30 μg administered to the subject a total of two times.

In some embodiments, the method includes administering to the subject a saRNA composition described herein at a dosage of between 10 μg/kg and 400 μg/kg is administered to the subject. In some embodiments the dosage of the saRNA polynucleotide is 1 to 5 μg, 5 to 10 μg, 10 to 15 μg, 15 to 20 μg, 10 to 25 μg, 20 to 25 μg, 20 to 50 μg, 30 to 50 μg, 40 to 50 μg, 40 to 60 μg, 60 to 80 μg, 60 to 100 μg, 50 to 100 μg, 80 to 120 μg, 40 to 120 μg, 40 to 150 μg, 50 to 150 μg, 50 to 200 μg, 80 to 200 μg, 100 to 200 μg, 120 to 250 μg, 150 to 250 μg, 180 to 280 μg, 200 to 300 μg, 50 to 300 μg, 80 to 300 μg, 100 to 300 μg, 40 to 300 μg, 50 to 350 μg, 100 to 350 μg, 200 to 350 μg, 300 to 350 μg, 320 to 400 μg, 40 to 380 μg, 40 to 100 μg, 100 to 400 μg, 200 to 400 μg, or 300 to 400 μg per dose. In some embodiments, the saRNA composition is administered to the subject by intradermal or intramuscular injection. In some embodiments, the saRNA composition is administered to the subject on day zero. In some embodiments, a second dose of the saRNA composition is administered to the subject on day twenty-one.

In some embodiments, the subject is about 5 years old or younger. For example, the subject may be between the ages of about 1 year and about 5 years (e.g., about 1, 2, 3, 5 or 5 years), or between the ages of about 6 months and about 1 year (e.g., about 6, 7, 8, 9, 10, 11 or 12 months). In some embodiments, the subject is about 12 months or younger (e.g., 12, 11, 10, 9, 8, 7, 6, 5, 4, 3, 2 months or 1 month). In some embodiments, the subject is about 6 months or younger.

In some embodiments, the subject was born full term (e.g., about 37 to 42 weeks). In some embodiments, the subject was born prematurely, for example, at about 36 weeks of gestation or earlier (e.g., about 36, 35, 34, 33, 32, 31, 30, 29, 28, 27, 26 or 25 weeks). For example, the subject may have been born at about 32 weeks of gestation or earlier. In some embodiments, the subject was born prematurely between about 32 weeks and about 36 weeks of gestation. In such subjects, a RNA (e.g., mRNA) vaccine may be administered later in life, for example, at the age of about 6 months to about 5 years, or older.

In some embodiments, the subject is a young adult between the ages of about 20 years and about 50 years (e.g., about 20, 25, 30, 35, 40, 45 or 50 years old).

In some embodiments, the subject is an elderly subject about 60 years old, about 70 years old, or older (e.g., about 60, 65, 70, 75, 80, 85 or 90 years old).

In some embodiments, the subject has been exposed to influenza (e.g., C. trachomatis); the subject is infected with influenza (e.g., C. trachomatis); or subject is at risk of infection by influenza (e.g., C. trachomatis).

In some embodiments, the subject has been exposed to betacoronavirus (e.g., SARS-CoV-2); the subject is infected with betacoronavirus (e.g., SARS-CoV-2); or subject is at risk of infection by betacoronavirus (e.g., SARS-CoV-2).

In some embodiments, the subject has received at least one dose of an immunogenic composition against betacoronavirus (e.g., SARS-CoV-2), e.g., selected from any one of COMIRNATY®, the Pfizer-BioNTech COVID-19 vaccine, the Moderna mRNA-1273 COVID-19 vaccine, and the Janssen COVID-19 vaccine; the subject has received at least two doses of an immunogenic composition against betacoronavirus (e.g., SARS-CoV-2); the subject is receiving at least one dose of an immunogenic composition against betacoronavirus (e.g., SARS-CoV-2), e.g., selected from any one of COMIRNATY®, the Pfizer-BioNTech COVID-19 vaccine, the Moderna mRNA-1273 COVID-19 vaccine, and the Janssen COVID-19 vaccine; or the subject is being administered an immunogenic composition against betacoronavirus (e.g., SARS-CoV-2), e.g., selected from any one of COMIRNATY®, the Pfizer-BioNTech COVID-19 vaccine, the Moderna mRNA-1273 COVID-19 vaccine, and the Janssen COVID-19 vaccine at risk of infection by betacoronavirus (e.g., SARS-CoV-2) concomitantly, simultaneously, or within 12 to 48 hours of any one of the immunogenic compositions against influenza disclosed herein.

In some embodiments, the subject is immunocompromised (has an impaired immune system, e.g., has an immune disorder or autoimmune disorder).

### Kits

Another aspect of the disclosure provides kits comprising the compositions as disclosed herein. A kit may include, in addition to the composition of the disclosure, diagnostic or therapeutic agents. A kit may also include instructions for use in a diagnostic or therapeutic method. In some embodiments, the kit includes a composition of the disclosure and a diagnostic agent.

In yet another embodiment, the disclosure comprises kits that are suitable for use in performing the methods of treatment described herein. In one embodiment, the kit contains a first dosage form comprising one or more of the compositions of the disclosure in quantities sufficient to carry out the methods of the disclosure. In another embodiment, the kit comprises one or more compositions of the disclosure in quantities sufficient to carry out the methods of the disclosure and a container for the dosage.

### EXAMPLES

The present disclosure is described in greater detail by way of specific examples. The following examples are offered for illustrative purposes and are not intended to limit the disclosure in any manner.

### Example 1: Small Molecule Modulators of Innate Immunity of saRNA Encoding Influenza Antigen

### Introduction

The saRNA vaccine is based on a synthetic RNA derived from Venezuelan Equine Encephalitis replicon and delivered in a lipid nanoparticle (LNP) formulation. saRNA can generate many copies of antigen-encoding RNA as well as double-stranded RNA (dsRNA) intermediates in the target cell, leading to high and prolonged expression of the antigen and additional self-adjuvanting of the innate immune response. The innate immune response elicited by saRNA, however, needs to be appropriately balanced to potentiate the adaptive immune response, as excessive innate activation could have a detrimental effect on the launch of synthetic RNA, viability of transfected cells, and consequent development of the adaptive immune responses.

One potential strategy to reduce detrimental effects of innate immune activation on saRNA vaccines is the co-administration of small molecules that inhibit key signaling sensors/mediators' activities in the type I IFN pathway. We characterized how co-formulation with small molecules targeting interleukin-1 receptor-associated kinase 4 (IRAK4), a key signal transducing adaptor of interleukin (IL)-1 family receptors and Toll-like receptor (TLR) signaling or those targeting cytoplasmic RNA sensors including retinoic acid inducible gene 1(RIG-1), affects gene of interest (GOI) expression in vitro in human cells. We found that IRAK4 modulators and compounds that specifically modulate the signaling activity of RIG-1 enhance protein expression of saRNA in vitro. While a similar strategy was applied to target the JAK/STAT pathway in the downstream IFN response (Blakney et al., 2021), the saRNA improvement mediated by this class of small molecules targeting the upstream of the type I IFN pathway has not been previously described.

### Methods

1. Screening Immunity Modulating Compounds for Effects on saRNA Expression and Replication

In order to identify small molecules capable of modulating innate immunity, a high-throughput small-molecule screening strategy was designed and optimized on the basis of the HiBiT luminescent reporter system, which was chosen because of its high sensitivity and stability. The HiBiT tag was added to the 3′ end of the saRNA-HA mRNA encoding construct to create the saRNA-HA-HiBiT construct. The saRNA-HA-HiBiT mRNA was produced by in vitro transcription, then formulated into LNPs.

Tested compounds were solubilized in DMSO at 30 mM and diluted as follows: Seven×2-fold dilutions were made by starting with 20 μL of 30 mM stock. 10 μL+10 μL DMSO for each dilution across two 96 well v-bottom plates. Test range 30 μM, 15 μM, 7.5 μM, 3.75 μM, 1.875 μM, 0.9375 μM, 0.4688 μM, 0 μM. DMSO was included as the control. The compound dilutions were transferred at a 384 LDV Echo plate (Labcyte) and 25 nL/well of each dilution was transferred to Greiner #781080 white/solid assay plates using the Labcyte Echo 550 acoustic liquid dispenser. Three to six replicates were performed per concentration.

Hela cells were cultured in MEM+1× Earls Salts media (Gibco #1195080)+1× Non-essential Amino Acids, 1× Glutamax+10% FBS (Certified OneShot ThermoFisher). 6000 cells per well in 20 μL was plated into the assay plates containing the compounds using a Multi-Drop Combi dispenser (ThermoFisher).

saRNA-HA-HiBiT LNPs were diluted to the appropriate concentration (0.5 to 5 ng/μL) in Tris/Sucrose buffer, and 5 μL per well was added using the Multi-Drop Combi dispenser. The plates were spun for 5 min at 1K, then incubated 24 to 48 hours at 37° C. 5% CO₂. The following morning HiBiT lytic reagent was made up (11 mls for each plate) according to the manufacturer's specification: 11 mL HiBiT Lytic Buffer+110 μL LgBiT protein (1:100)+220 μL substrate (1:50). 25 μL HiBiT Lytic reagent was added per well using the Multi-Drop Combi dispenser and the plates spun 5 minutes at 1000 rpm. The plates were incubated in the dark for a total of 10 minutes, then read for total luminescence on the Perkin-Elmer Envision plate reader. The data was exported and analyzed/graphed in Excel and/or GraphPad Prism. Percent control was calculated by dividing the average LCPS value of the 6 wells by the average DMSO value×100.

### 2. Cell-Based Assays for Assessing Effects of IRAK4 or RIG-1 Compounds on Gene of Interest (GOI) Transcription and Translation as Well as Cellular Toxicity

Hela cells were used to test the influence of compounds on saRNA GOI expression while simultaneously reporting on relative toxicity by flow cytometry. In addition, we evaluated the effects of IRAK4/RIG-I compounds on saRNA replicase and GOI transcript levels via qRT-PCR. Hela cells were grown and maintained in T75 flasks or two-chamber CellSTACKs containing Dulbecco's Modified Eagle Medium, DMEM (Giboco) supplemented with 10% heat-inactivated fetal bovine serum (HI-FBS) (Gibco) and 1 IU/mL of penicillin, 1 mg/mL of streptomycin (Hela growth media).

For the in vitro HA antigen expression assays, 0.5 mL of cells at 220,000 cells/mL were placed (seeded) in 24-well plates in Hela growth media, but with 10% non-heat inactivated FBS (non-HI FBS) in lieu of HI-FBS (Hela transfection media). Each compound was dissolved from a crystalline solid to a final concentration of 10 μM in 100% DMSO. Four-point, 2-fold dilution series of each compound, or the vehicle control, were performed in Opti-MEM (Gibco) to generate 2× desired concentrations for cell treatment. LNPs encapsulating saRNA-TC83-A/Wisc/588/19 HA-40A were diluted to a concentration of 250 ng/50 μL in Opti-MEM and mixed 1:1 with the 2× drug dilutions or vehicle control, respectively. 50 μL of each saRNA:compound mix, or compounds without saRNA, were then added, dropwise, to their respective wells. After 20 to 24 hours, cells were harvested for flow cytometric analysis of HA protein expression in the following manner: Hela transfection media was aspirated from each well, and 205 μL of StemPro Accutase (Gibco) was added to each well. Cells were then incubated for 4 to 8 minutes at 37° C. with 5% CO₂. Following the incubation, cells were transferred to 96-well U-bottomed plates and centrifuged at 600 to 800 g, for 5 minutes at 4° C. After centrifugation, the Accutase was aspirated from the cell pellets and cells were washed with 250 μL of PBS with calcium and magnesium (Corning), prior to staining with LIVE/DEAD Fixable Aqua Dead Cell Stain (Invitrogen) for 30 minutes at room temperature. Cells were then washed with PBS containing calcium and magnesium (Corning) before being fixed and permeabilized by adding 150 μL/well of BD Cytofix/Cytoperm solution and incubating on ice for 1 hour. The Cytofix/Cytoperm solution was then aspirated, and cells were washed using a 10×BD Perm/Wash solution diluted to 1× in sterile, nuclease-free water. Following the wash, the primary antibody (Pfizer generated F16) was diluted 1:1000 in 1×BD Perm/Wash and 100 μL was added to each well before a 1 hour incubation on ice. Once the 1 hour primary incubation was complete, cells were washed with 1×BD Perm/Wash and then stained with a goat-anti-human AF488 secondary antibody (Invitrogen) at a 1:500 dilution in 1×BD Perm/Wash buffer. After a 1 hour incubation on ice, the secondary antibody dilution was aspirated, and cells were washed with 1×BD Perm/Wash. Next, the cells were washed 2× in DPBS (Gibco) supplemented with 2% bovine serum albumin (BSA) (FACS Buffer), prior to a final resuspension in 200 μL/well of FACS Buffer. Samples were acquired on an LSRFortessa (BD Biosciences). Analysis was performed using FlowJo software.

We evaluated the effects of the IRAK4/RIG-1 compounds in Hela cells, on saRNA replication by assaying saRNA replicase and GOI transcript levels via qRT-PCR. For the assay, 0.5 mL of cells at 220,000 cells/mL were seeded in 24-well plates using Hela transfection media. Compound and saRNA dilutions were performed as described for the HA antigen expression assay and added to cells in a dropwise manner. After 20 to 24 hours, cell lysates were harvested using 205 μL/well of TriZol reagent (Invitrogen) and were stored at −80° C. until total RNA isolation was performed using the Direct-zol-96 RNA isolation kit with an in-column Dnase treatment (Zymo Research). RT-qPCR was performed and relative transcript levels of saRNA replicase and GOI were measured using the Luna Universal One-Step RT-qPCR kit (NEB) with the following parameters: A 20 μL final volume/well mix containing 0.5 μM of each forward and reverse primer, 2 μL of total RNA, 50 nM Rox Dye (ThermoFisher), 1 μL of Luna warm-start RT mix (10×) (NEB), 10 μL Luna one-step RT-qPCR mix (2×) (NEB), and 6.7 μL of nuclease-free water (Invitrogen) was added to either 384 or 96-well optical plates (Applied Biosystems). Amplification was conducted in an QuantStudio 3 or QuantStudio 5, Real-Time PCR machine under the thermal profile of 55° C. for 10 minutes then 95° C. for 1 minute, followed by 40 cycles of 95° C. for 10 seconds and 60° C. for 1 minute. Each sample was analyzed in triplicate and normalized via the dCt method to the level of human ribosomal protein S11 mRNA. The primers used for quantitative one-step real-time PCR were: nsp2, Ha, and Rps11.

### Results

### 1. IRAK4 Modulators

Targeted IRAK4 degraders are bifunctional compounds that bind IRAK4 with one arm and the other consists of an immunomodulatory imide (IMiD) that recruits the E3 ligase complex component cereblon. A ubiquitin E3 ligase is an enzyme involved in marking proteins for ubiquitination and subsequent destruction by the proteasome. Zimlovisertib (PF-06650833) is a potent, selective and orally active inhibitor of interleukin-1 receptor associated kinase 4 (IRAK4) with IC50s of 0.2 and 2.4 nM in the cell and PBMC assay, respectively. Zimlovisertib is used to treat diseases such as rheumatoid arthritis, lupus, and lymphomas (Lee et al., J Med Chem. July 13; 60(13), pp. 5521-5542 (2017)). It has also been studied for the treatment of Covid-19 pneumonia (Zapata-Acevedo et al., ChemPhysChem 2022, 23; https://doi.org/i0.1002/cphc.202200455).

We characterized how co-delivery of antigen-encoding saRNA with an IRAK4 degrader or Zimlovisertib in vitro in human cells affects: 1) cell viability; 2) the number of cells expressing antigen encoded by saRNA and geometric mean fluorescence intensity (GMFI) of the antigen in saRNA-transfected cells, which reflects the antigen copy number and thus the translational rate of the encoding RNA; and 3) saRNA replication.

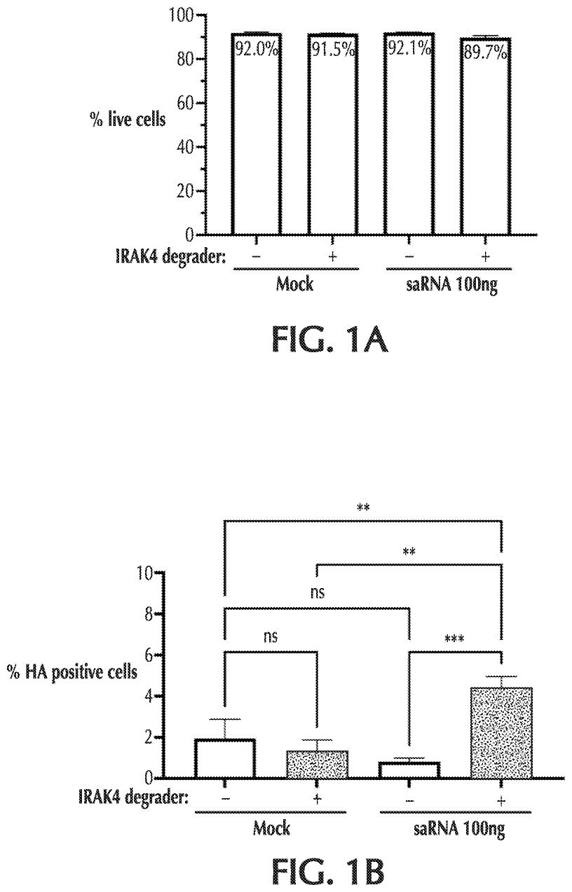

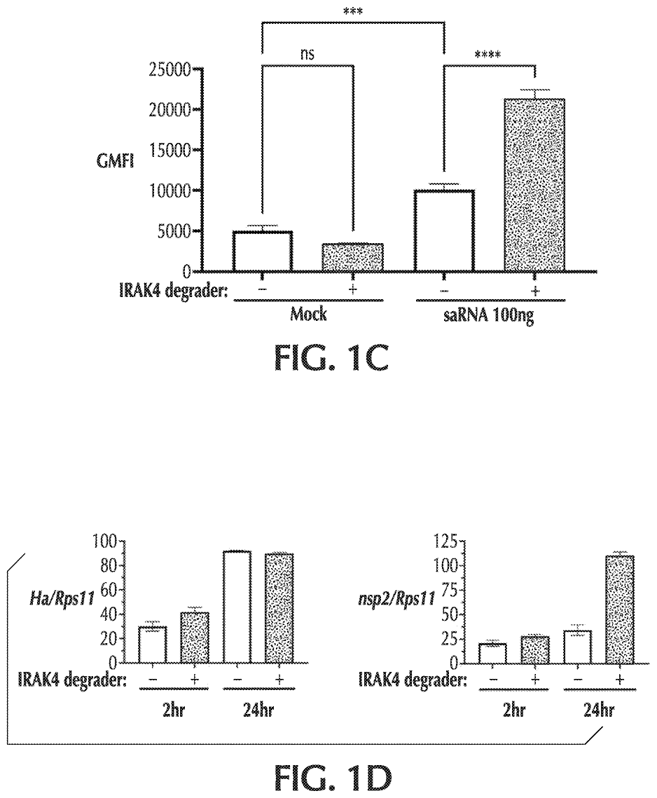

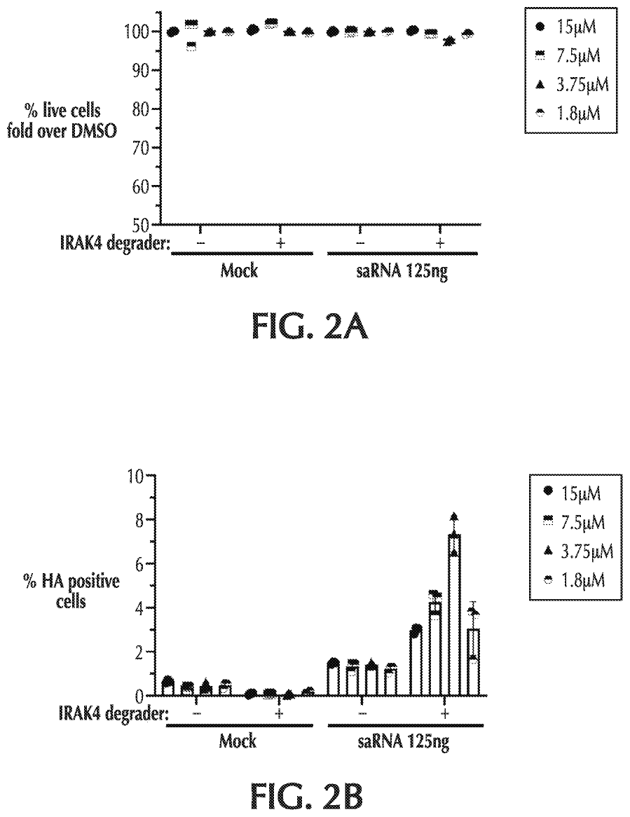

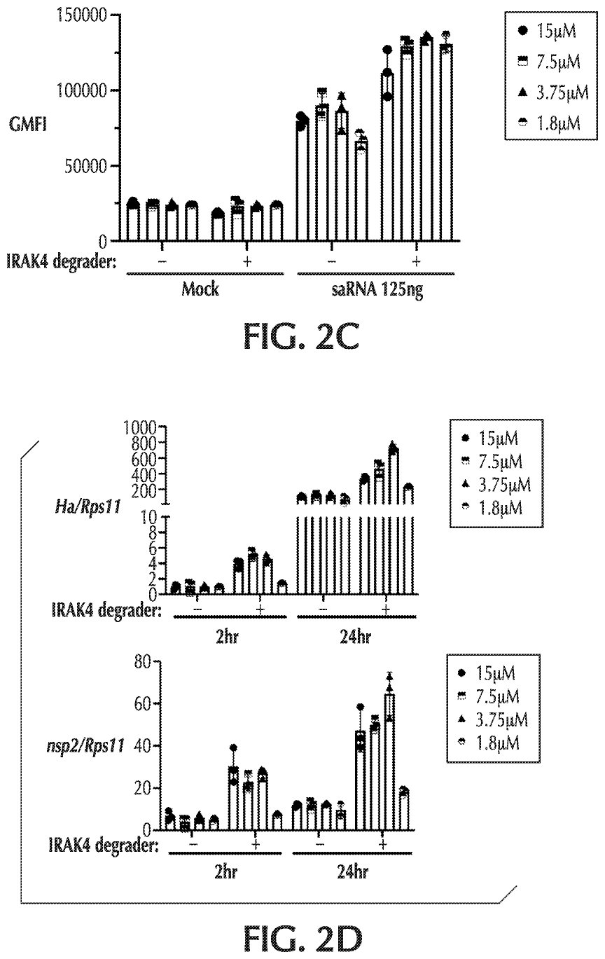

Upon introduction of the IRAK4 targeting compound, PF-07911620, into the growth medium, we observed potent inhibition of IRAK4 signaling resulting in significantly higher frequency of HA-positive cells than vehicle controls (5.5-fold, FIG. 1B). In addition, a 1.8-fold increase of the HA GMFI was achieved by PF-07911620 co-transfection compared to transfecting with saRNA alone (FIG. 1C). In addition to an increased translation, a trend toward greater levels of plus-stranded saRNA was also observed in the presence of PF-07911620. Despite a significant increase in HA expression, cell growth and viability were unaffected by the compound (FIG. 1A). Upon treatment with PF-07932784, another IRAK4 degrader, or PF-06650833, a closely related inhibitor, a similar although less pronounced improvement was found (FIG. 2, data not shown).

### 2. RIG-1 Modulators

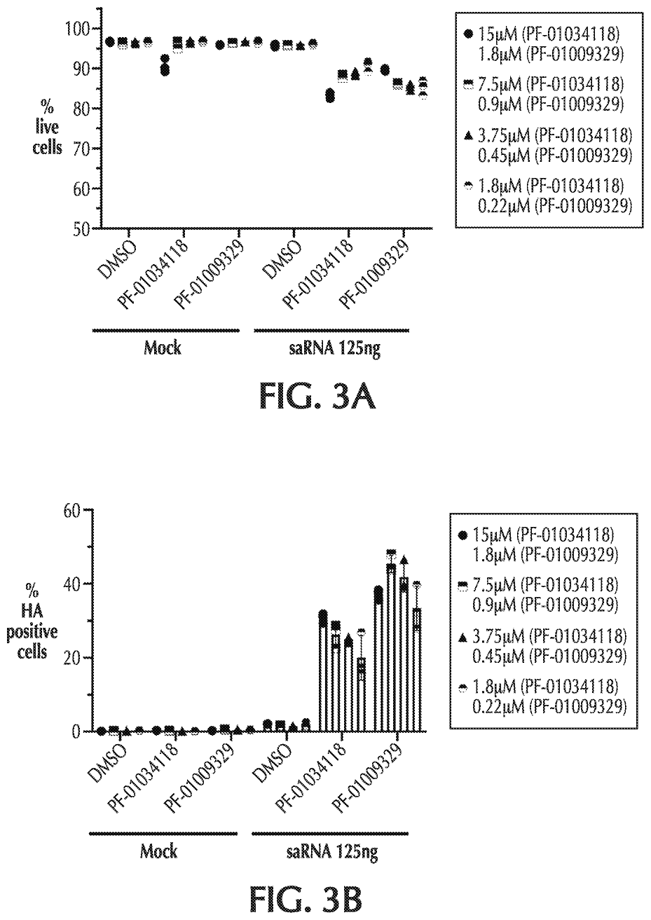

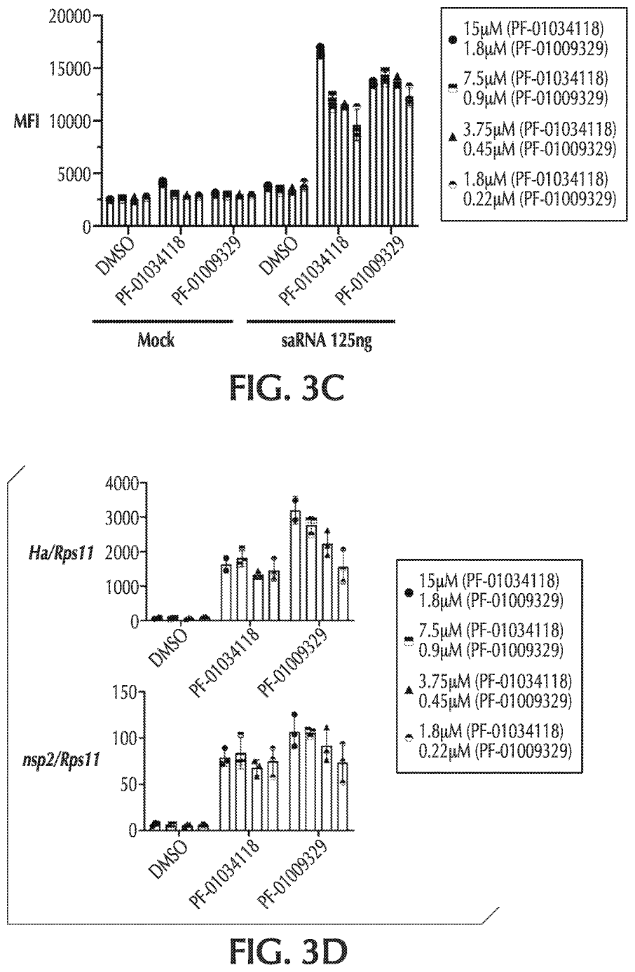

To examine the influence of the compounds on RIG-1 (or other RNA sensors)-dependent IFN induction, we treated Hela cells with a range of PF-01034118 & PF-01009329 concentrations with HA encoding saRNA which initiate the IFN signaling pathway. Treatment with PF-01034118 or PF-01009329 significantly increased the percentage of antigen-positive cells, as much as 28-fold (saRNA with PF-01009329 at 0.9 mM vs. saRNA with DMSO control) or 14.1-fold (saRNA with PF-01034118 at 15 mM vs. saRNA with DMSO control). Both compounds significantly increased transcript levels of both the antigen (Ha, 28-fold saRNA with PF-01034118 at 7.5 mM or 43-fold saRNA with PF-01009329 at 1.8/0.9 mM compared to saRNA with DMSO control) and the replicase (nsP2), with the PF-01009329 molecule demonstrating the greatest overall increases in transcript levels. Consistent with the initial compound screen based on the Hibit luminescent reporter system, we observed a potent effect of PF-01009329 on both HA antigen expression and saRNA transcription at much lower concentration than that of PF-01034118 (FIG. 3). Importantly, compound-specific cell cytotoxicity was not observed except at the highest PF-01034118 concentration (15 mM) tested.

### Example 2: Small Molecule Innate Immunity Modulators of saRNA and Small Molecules that Facilitate Efficient RNA Delivery into Target Cells

### Introduction

The saRNA vaccine is based on a synthetic RNA derived from Venezuelan Equine Encephalitis replicon and delivered in a lipid nanoparticle (LNP) formulation. saRNA can generate many copies of antigen-encoding RNA as well as double-stranded RNA (dsRNA) intermediates in the target cell, leading to high and prolonged expression of the antigen and additional self-adjuvating of innate immune responses. The innate immune responses elicited by saRNA, however, need to be appropriately balanced to potentiate adaptive immune responses, as excessive innate activation could have a detrimental effect on the launch of synthetic RNA, viability of transfected cells, and consequently development of the adaptive immune responses.

One potential strategy to reduce detrimental effects of innate immune activation on saRNA vaccines is the co-administration of small molecules that inhibit key signaling sensors/mediators' activities in the type I IFN pathway. We characterized how co-formulation with small molecules targeting interleukin-1 receptor-associated kinase 4 (IRAK4), a key signal transducing adaptor of interleukin (IL)-1 family receptors and Toll-like receptor (TLR) signaling affects gene of interest (GOI) expression in vitro in human cells. We found that IRAK4 degraders or inhibitors and compounds with a 6,7-dimethoxy-2-(1-piperazinyl)-4-quinazolinamine (DPQ) scaffold that interacts with the major groove of RNA at the (A-A)-U internal loop (Lee et al., Chem. Commun. 50:368-370 (2014)) enhance protein expression of saRNA in vitro. While a similar strategy was applied to target the JAK/STAT pathway in the downstream IFN response (Blakney et al., Mol. Ther. March 3; 29(3): 1174-1185 (2021)), we believe saRNA improvement mediated by this class of small molecules targeting the upstream of the type I IFN pathway has not been previously described.

### Methods

### 1. Identification of IRAK4 Antagonists & DPQ Compounds

In order to identify small molecules capable of modulating innate immunity, a high-throughput small-molecule screening strategy was designed and optimized on the basis of the Hibit luminescent reporter system, which was chosen because of its high sensitivity and stability.

### 2. Cell-Based Assays for Assessing Effects of IRAK4 or DPQ Compounds on Gene of Interest (GOI) Transcription and Translation as Well as Cellular Toxicity

HeLa cells were used to test the influence of compounds on modRNA or saRNA GOI expression while simultaneously reporting on relative toxicity by flow cytometry. In addition, we evaluated the effects of IRAK4/DPQ compounds on saRNA replicase and gene of interest (GOI) transcript levels via RT-qPCR.

HeLa cells were grown and maintained in T75 flasks or two-chamber CellSTACKs containing Dulbecco's Modified Eagle Medium, DMEM (Giboco) supplemented with 10% heat-inactivated fetal bovine serum (HI-FBS) (Gibco) and 1 IU/mL of penicillin, 1 mg/mL of streptomycin (HeLa growth media).

For the in vitro HA or NA antigen expression assays, 0.5 mL of cells at 220,000 cells/mL were placed (seeded) in 24-well plates in HeLa growth media, but with 10% non-heat inactivated FBS (non-HI FBS) in lieu of HI-FBS (HeLa transfection media). Each compound was dissolved from a crystalline solid to a final concentration of 10 mM in 100% DMSO. Four or eleven-point, 2-fold dilution series of each compound, or the vehicle control, were performed in Opti-MEM (Gibco) to generate 2× desired concentrations for cell treatment. LNPs encapsulating modRNA-A/Wisc/588/19 HA-80A (RMM71), saRNA-TC83-A/Wisc/588/19 HA-40A (RMM59) or bicistronic saRNA-TC83-A/Wisc/588/19 HA-NA-80A with or without 50% m1Y and/or 100% m5C incorporation were diluted to a concentration of 52-250 ng/50 mL in Opti-MEM and mixed 1:1 with the 2× drug dilutions or vehicle control, respectively. 50 mL of each modRNA or saRNA:compound mix, or compounds without modRNA or saRNA, were then added, dropwise, to their respective wells. After 20-24 hours cells were harvested for flow cytometric analysis of HA or NA protein expression in the following manner: HeLa transfection media was aspirated from each well, and 205 mL of StemPro Accutase (Gibco) was added to each well. Cells were then incubated for 4-8 minutes at 37° C. with 5% CO₂. Following the incubation, cells were transferred to 96-well U-bottomed plates and centrifuged at 600-800 g, for 5 minutes at 4° C. After centrifugation, the Accutase was aspirated from the cell pellets and cells were washed with 250 mL of PBS with calcium and magnesium (Corning), prior to staining with LIVE/DEAD Fixable Aqua Dead Cell Stain (Invitrogen) for 30 minutes at room temperature. Cells were then washed with PBS containing calcium and magnesium (Corning) before being fixed and permeabilized by adding 150 mL/well of BD Cytofix/Cytoperm solution and incubating on ice for 1 hour. The Cytofix/Cytoperm solution was then aspirated, and cells were washed using a 10×BD Perm/Wash solution diluted to 1× in sterile, nuclease-free water. Following the wash, the primary antibody (Pfizer generated F16 or polyclonal Ab) was diluted 1:1000 in 1×BD Perm/Wash and 100 mL was added to each well before a 1 hour incubation on ice. Once the 1 hour primary incubation was complete, cells were washed with 1×BD Perm/Wash and then stained with a goat-anti-human or rabbit AF488 secondary antibody (Invitrogen) at a 1:500 dilution in 1×BD Perm/Wash buffer. After a 1 hour incubation on ice, the secondary antibody dilution was aspirated, and cells were washed with 1×BD Perm/Wash. Next, the cells were washed 2× in DPBS (Gibco) supplemented with 2% bovine serum albumin (BSA) (FACS Buffer), prior to a final resuspension in 200 mL/well of FACS Buffer. Samples were acquired on an LSRFortessa (BD Biosciences). Analysis was performed using FlowJo software.

We evaluated the effects of the IRAK4/DPQ compounds in HeLa cells, on saRNA replication by assaying saRNA replicase and GOI transcript levels via qRT-PCR. For the assay, 0.5 mL of cells at 220,000 cells/mL were seeded in 24-well plates using HeLa transfection media. Compound and modRNA or saRNA dilutions were performed as described for the HA antigen expression assay and added to cells in a dropwise manner. After 20 to 24 hours, cell lysates were harvested using 205 mL/well of TriZol reagent (Invitrogen) and were stored at −80° C. until total RNA isolation was performed using the Direct-zol-96 RNA isolation kit with an in-column Dnase treatment (Zymo Research). RT-qPCR was performed and relative transcript levels of saRNA replicase and GOI were measured using the Luna Universal One-Step RT-qPCR kit (NEB) with the following parameters: A 20 μL final volume/well mix containing 0.5 mM of each forward and reverse primer, 2 mL of total RNA, 50 nM Rox Dye (ThermoFisher), 1 mL of Luna warm-start RT mix (10×) (NEB), 10 μL Luna one-step RT-qPCR mix (2×) (NEB), and 6.7 mL of nuclease-free water (Invitrogen) was added to either 384 or 96-well optical plates (Applied Biosystems). Amplification was conducted in an QuantStudio 3 or QuantStudio 5, Real-Time PCR machine under the thermal profile of 55° C. for 10 minutes then 95° C. for 1 minute, followed by 40 cycles of 95° C. for 10 seconds and 60° C. for 1 minute. Each sample was analyzed in triplicate and normalized via the ΔCt method to the level of human ribosomal protein S11 mRNA. The primers used for quantitative one-step real-time PCR were: nsp2, Ha, and Rps11.

### 3. Influenza Microneutralization Assay

The influenza virus microneutralization assay (MNT) quantitatively measures functional antibodies in serum that neutralize influenza virus activity, preventing productive infection of a host cell monolayer. A neutralization reaction occurs when influenza virus is incubated with serum samples; this reaction mixture is then applied to a monolayer of Madin-Darby Canine Kidney (MDCK) cells to measure the extent of neutralization. MNT titers are reported as the reciprocal of the dilution that results in 50% or 90% reduction in infection when compared to a no serum control. The 1-Day MNT measures anti-HA neutralizing antibodies.

### 4. Mode of Action Studies for PF-01009329

a. Time Course Assay

One day prior to beginning the assay, HeLa cells were seeded in 24-well plates (Corning) at a density of 1.1×10⁵ cells/well and incubated overnight with 5% CO₂ at 37° C. PF-01009329, previously diluted to a 10 mM stock in 100% DMSO, was diluted in Opti-MEM so that the final concentration of small molecule with cells would be 0.2 mM. One hour prior to LNP addition, cells were pre-treated with PF-01009329 (pre-treat) or vehicle control (post-treat/co-treat) and spun at 600 g, for 5 minutes, prior to being placed back into the incubator for 1 hour. modRNA-LNP dilutions were then generated at a 2× final concentration of 26 ng/25 mL. For the co-treatment condition, 2×RNA-LNPs were mixed 1:1 with “2×” PF-01009329 and added directly to cells. RNA-LNPs were diluted 1:1 in Opti-MEM and added to cells for the pre-treat and post-treat conditions, to be consistent with the ad-mixture dosing used in the co-treatment condition. Following LNP addition to cells, the samples were centrifuged at 600 g for 5 minutes and placed back into the incubator. One hour after LNP addition to cells, PF-01009329 was added to the post-treatment wells, while vehicle was used for all other conditions. Cells were then placed back in the incubator at 37° C. for the indicated incubation times. Cell lysates were collected by decanting and disposing of the cell supernatants, at the indicated timepoints, followed by addition of Trizol (Invitrogen) reagent directly to cells. Lysates were incubated for 5 minutes at room temperature before being transferred to U-bottomed, 96-well plates (Falcon), and stored at −80° C. until RNA extraction and RT-qPCR analysis as described in Method 2.

b. Single-Molecule RNA Fluorescence In Situ Hybridization (FISH) Method to Assess the Internalization of saRNA-LNP

Cells incubated with saRNA-LNP with or without PF-01009329 were washed with PBS, fixed with freshly prepared 4% paraformaldehyde for 10 minutes, quenched, washed three times with PBS, and permeabilized with 0.1% Triton X-100 for 10 minutes. After washing the cells with PBS, pre-hybridization buffer was added for 30 minutes. The supernatant was removed, and HA-CAL Fluor Red 590 Dye probe (Stellaris) was diluted in hybridization buffer (250 nM/well) and incubated with cells for 3 hours at 37° C. The supernatant was removed, washed by incubating pre-hybridization buffer for 10 min. Cells were then washed two times with PBS. Finally, cells were incubated with DAPI (0.5 μg/mL) to stain the nuclei and CellMask Deep Red (1/140,000) to stain the cytoplasm.

### Results

### 1. IRAK4 Degrader (PF-07911620 & PF-07932784, Kymera's IRAKIMiD) & IRAK4 Inhibitor (PF-06650833, Zimlovisertib)

Targeted IRAK4 degraders are bifunctional compounds that bind IRAK4 with one arm and the other consists of an immunomodulatory imide (IMiD) that recruits the E3 ligase complex component cereblon. A ubiquitin E3 ligase is an enzyme involved in marking proteins for ubiquitination and subsequent destruction by the proteasome.

Zimlovisertib (PF-06650833) is a potent, selective and orally active inhibitor of interleukin-1 receptor associated kinase 4 (IRAK4) with IC₅₀s of 0.2 and 2.4 nM in the cell and PBMC assay, respectively. Zimlovisertib is used to treat diseases such as rheumatoid arthritis, lupus, and lymphomas (Lee et al., J. Med. Chem. July 13:60(3):5521-5542 (2017)).

We characterized how co-delivery of antigen-encoding saRNA with small molecules IRAK4 degrader or Zimlovisertib in vitro in human cells affects: 1) cell viability, 2) the number of cells expressing antigen encoded by saRNA and geometric mean fluorescence intensity (GMFI) of the antigen in saRNA-transfected cells, which reflects the antigen copy number and thus the translational rate of the encoding RNA, and 3) saRNA replication.

Upon introduction of IRAK4 targeting compound, PF-07911620 into the growth medium, we observed potent inhibition of IRAK4 signaling resulting in significantly higher frequency of HA-positive cells than vehicle controls (5.5-fold, FIG. 1B). In addition, a 1.8-fold increase of the HA GMFI was achieved by PF-07911620 co-transfection compared to transfecting with saRNA alone (FIG. 1C). In addition to an increased translation, a trend toward greater levels of plus-stranded saRNA was also observed in the presence of PF-07911620. Despite a significant increase in HA expression, cell growth and viability were unaffected by the compound (FIG. 1A). Upon treatment with PF-07932784, another IRAK4 degrader, or PF-06650833, a very closely related inhibitor, a similar although less pronounced improvement was found (FIG. 2, data not shown).

### 2. DPQ Compounds (PF-01034118 & PF-01009329)

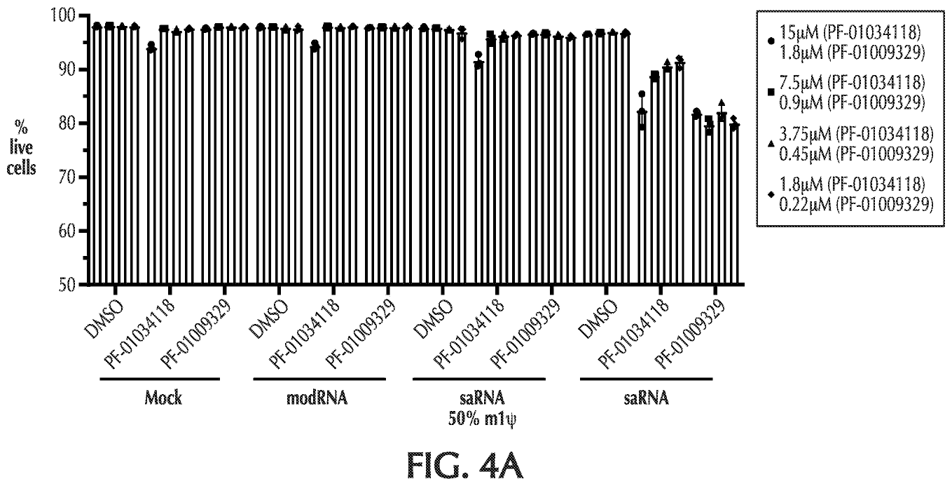

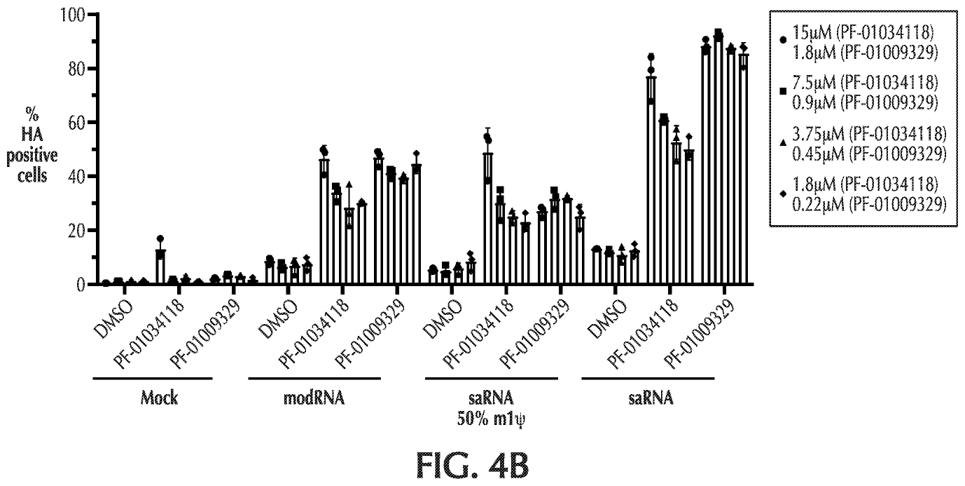

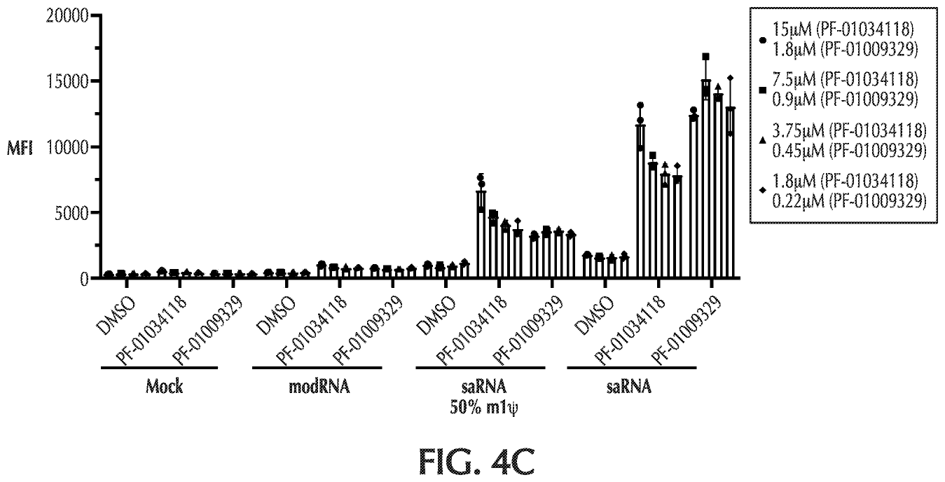

To examine the influence of the compounds on saRNA expression, we treated HeLa cells with a range of PF-01034118 & PF-01009329 concentrations with HA encoding saRNA. Treatment with PF-01034118 or PF-01009329 significantly increased the percentage of antigen-positive cells, as much as 28-fold (saRNA with PF-01009329 at 0.9 mM vs. saRNA with DMSO control) or 14.1-fold (saRNA with PF-01034118 at 15 mM vs. saRNA with DMSO control). Both compounds significantly increased transcript levels of both the antigen (Ha, 28-fold saRNA with PF-01034118 at 7.5 mM or 43-fold saRNA with PF-01009329 at 1.8/0.9 mM compared to saRNA with DMSO control) and the replicase (nsP2), with the PF-01009329 molecule demonstrating the greatest overall increases in transcript levels. Consistent with the initial compound screen based on the Hibit luminescent reporter system, we observed a potent effect of PF-01009329 on both HA antigen expression and saRNA transcription at much lower concentration than that of PF-01034118 (FIG. 3). Importantly, compound-specific cell cytotoxicity was not observed except at the highest PF-01034118 concentration (15 mM) tested. Notably, the effects of PF-01034118 at 15 mM and PF-01009329 at 1.8 mM on HA antigen expression from modRNA and (un)modified saRNA were similar (~6-fold increase) when rabbit polyclonal antibodies used to detect HA expression (FIG. 4).

3. IRAK4 Inhibitor (PF-06650833) vs. DPQ Compound (PF-01009329)

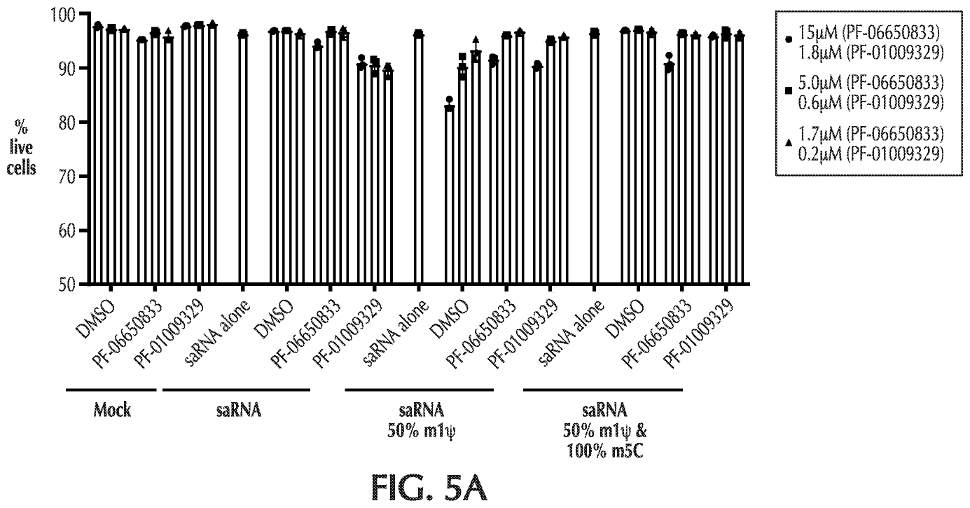

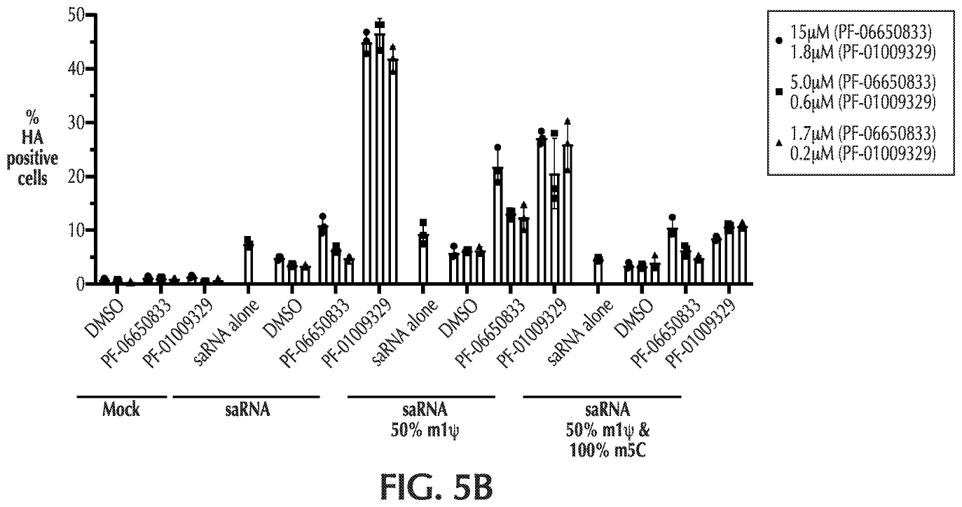

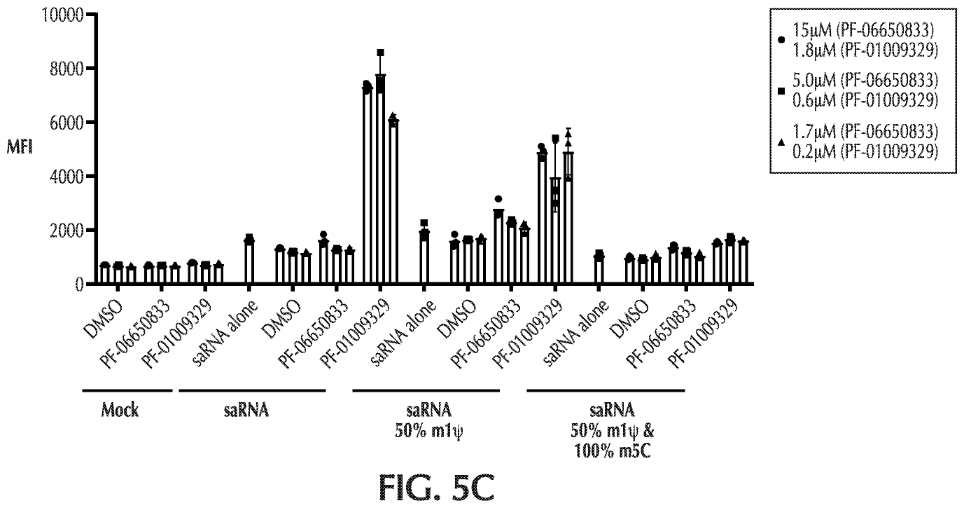

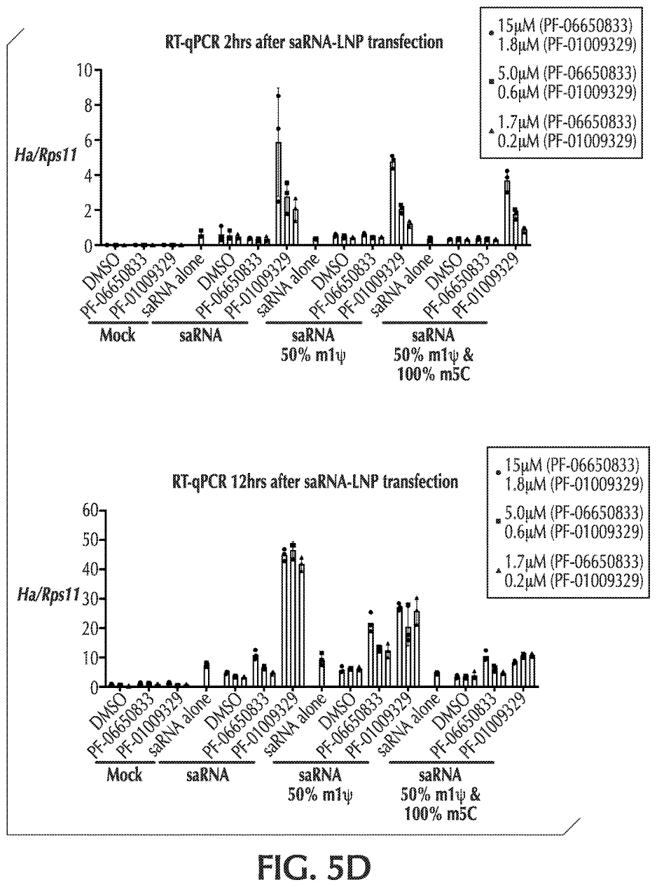

Co-treatment of HeLa cells with PF-06650833 and modRNA or bicistronic saRNA-LNPs increased the percentage of antigen-positive cells by 2.0-3.7-fold. PF-01009329, enhanced modRNA and bicistronic saRNA performance by up to 11-fold in HeLa, 4.6-fold in MDCK, and 16-fold in A549s, and 25-fold in THP-1, as measured by the percentage of antigen-positive cells (FIGS. 5 & 9, data not shown). Co-treatment of cells with modRNA or saRNA-LNPs and PF-01009329 but not PF-06650833 increased intracellular vaccine antigen transcripts in a dose-dependent manner by 2 hours after RNA-LNP addition, indicating the molecule may enhance LNP uptake (FIGS. 5D & 9D).

4. Co-Administered PF-01009329 Increased In Vitro Bicistronic saRNA Expression in LNPs with Varied Lipid Compositions (Cationic or Polymer Lipid)

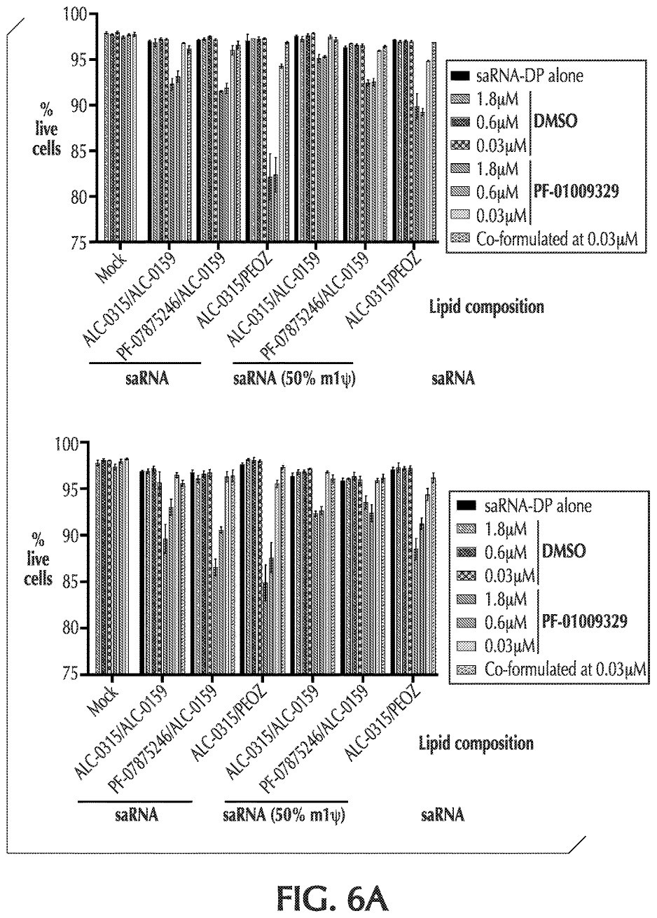

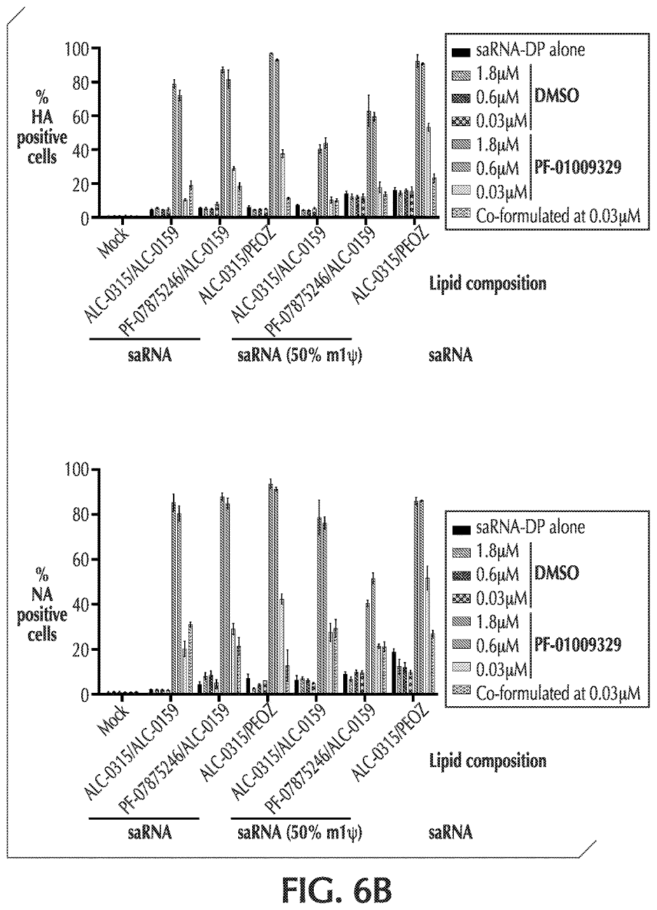

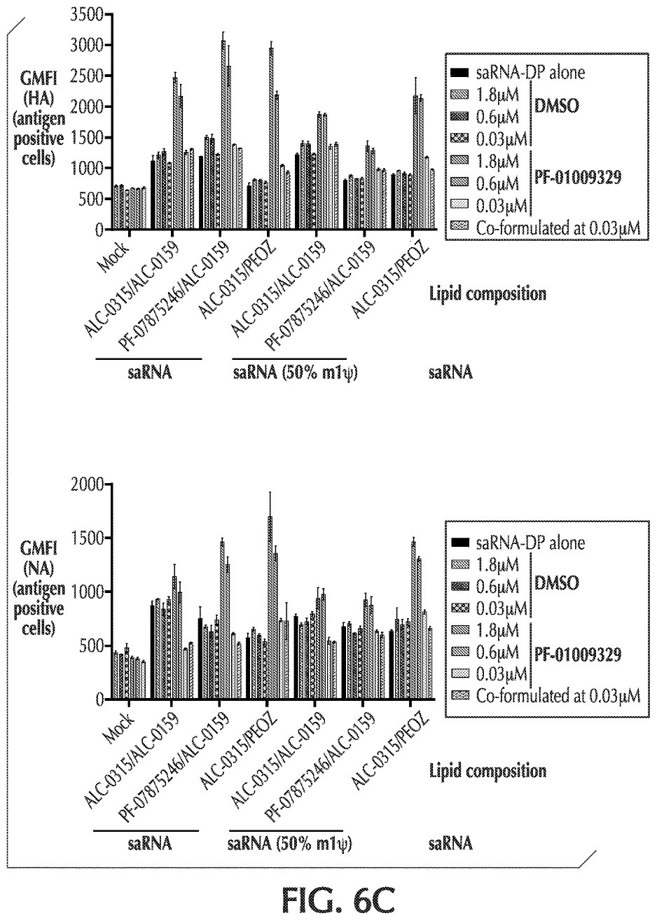

Baseline transfection efficiency, across the differential LNP compositions, demonstrated a trend of ALC-0315/PEOZ LNPs having higher transfection efficiency, as compared to the two other LNP compositions. saRNA generated with 50% m1ψ had transfection efficiencies which trended higher than those with saRNA carrying no modified nucleotides, with the exception that when NA was assayed, transfection efficiencies were mostly similar across saRNA with 50% m1ψ and without. Incorporation of PF-01009329, via co-formulation or admixture, increased transfection efficiency across all LNP compositions assayed, and both GOIs assayed (with exception of the co-formulated PF-07875246/ALC-0159, encapsulating saRNA with 50% m1ψ, with HA), highlighting the relatively broad capacity for this small molecule to improve RNA-LNP transfection efficiency. LNPs formulated with ALC-0315/ALC-0159 exhibited the greatest relative fold increase in cells expressing the antigen of interest as compared to the respective vehicle control, with lower relative fold increases observed when using LNPs with higher baseline transfection efficiencies. HA and NA expression, as measured via GMFI, was more variable than transfection efficiency, with a trend towards higher overall GOI expression when PF-01009329 was incorporated into LNPs. Cell viability correlated inversely with transfection efficiency and a greater reduction in viability was observed as the concentration of PF-01009329 was increased via admixture (FIG. 6). Taken together, these data highlight that the small molecule, PF-01009329, enhances transfection efficiency of LNPs across three differential formulation chemistries, and can do so regardless of the incorporation of 50% m1ψ uridine in the vaccine transcripts.

### 5. Immunogenicity

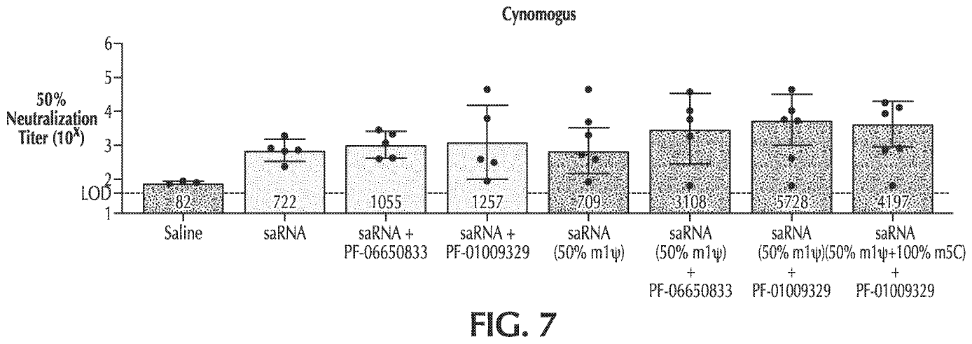

Intramuscular immunization of Cyno macaques with 2.5 μg of ALC-0315/PEOZ-formulated saRNA comprised of 0% m1ψ, 50% m1ψ or 50% m1ψ+100% m5C encoding the H1N1 A/Wisconsin/588/2019 HA-NA antigens and co-administered with small molecules (PF-06650833 or PF-01009329) induced a functional HA antibody response as measured by MNT (FIG. 7). Co-administration of either small molecule with unmodified saRNA-HA-NA resulted in a comparable immune response to saRNA-HA-NA vaccination alone at Day 42 (2 weeks post dose 2). Although 2 out of 5 animals in the saRNA-HA-NA+PF-01009329 group had neutralizing titers more than 8-fold higher than the saRNA group, the remaining 3 animals had lower titers, resulting in a geometric mean titer less than 2-fold higher than the saRNA-HA-NA alone. At Day 42, co-administration of PF-06650833 or PF-01009329 small molecule with 50% m1ψ modified saRNA-HA-NA improved immunogenicity over modified saRNA alone by 4 to 8-fold, respectively. Vaccination with saRNA-HA-NA incorporating 50% m1ψ and 100% m5C in combination with PF-01009329 resulted in a comparable neutralizing antibody response to 50% m1ψ saRNA-HA-NA+PF-01009329 2 weeks post dose 2.

6. PF-01009329: Crystalline Solid Form Vs. Hydrated Form

The total molar concentration achievable during co-formulation of the PF-010009329 with RNA was limited due to the relatively low aqueous solubility of the small molecule. In an attempt to increase the total amount of PF-01009329 which was incorporated into RNA-LNPs during co-formulation, a hydrate form of the small molecule was tested as opposed to the crystalline solid form of PF-01009329. Use of the hydrate form of PF-01009329 resulted in a co-formulation concentration increase of approximately 4-fold as compared to when the crystalline solid form of the small molecule was used, leading to a formulation concentration of 13.6 mg/mL, increased from 3.4 mg/ml, prior to LNP filtration. This LNP batch with higher PF-01009329 concentration was denoted “4×” in reference to the 4-fold higher target loading concentration of PF-01009329 in LNPs. In addition to the 4-fold loading increase, co-formulations were generated with 3-fold and 2-fold boosted PF-01009329 concentrations, and were labeled 3× and 2×, respectively, while the standard 3.4 mg/mL formulation was indicated as 1×.

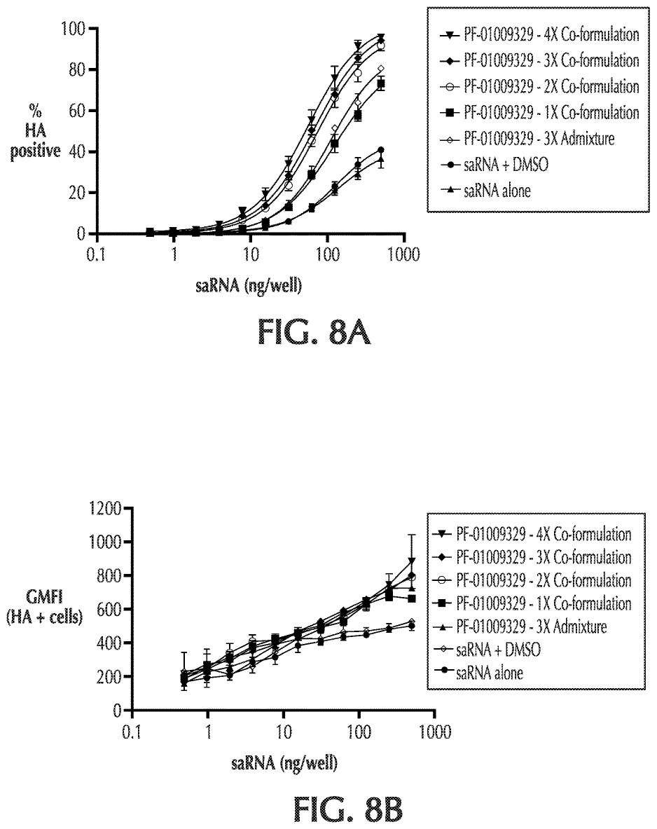

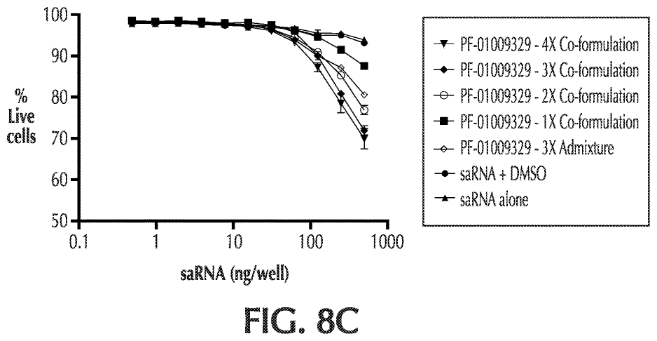

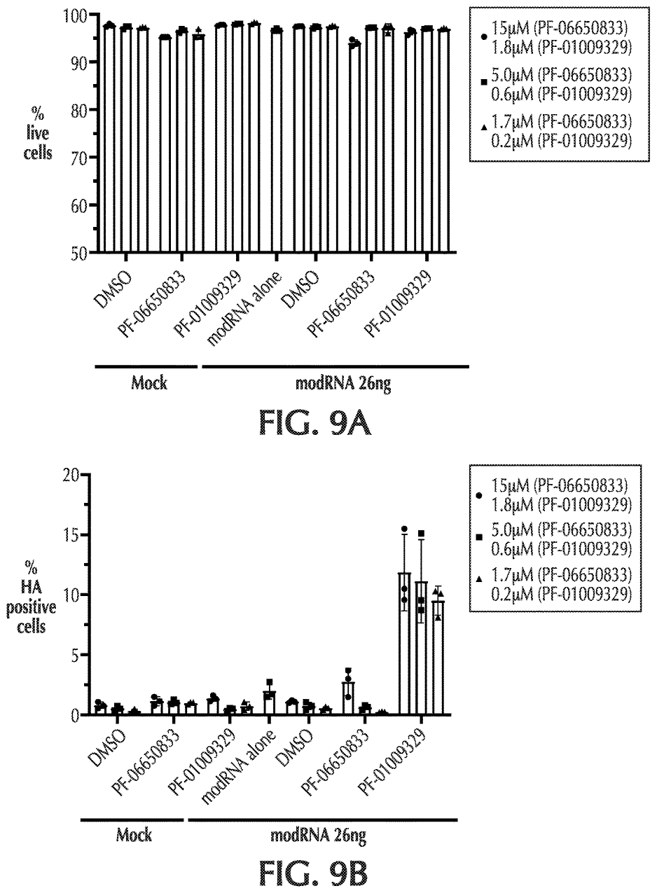

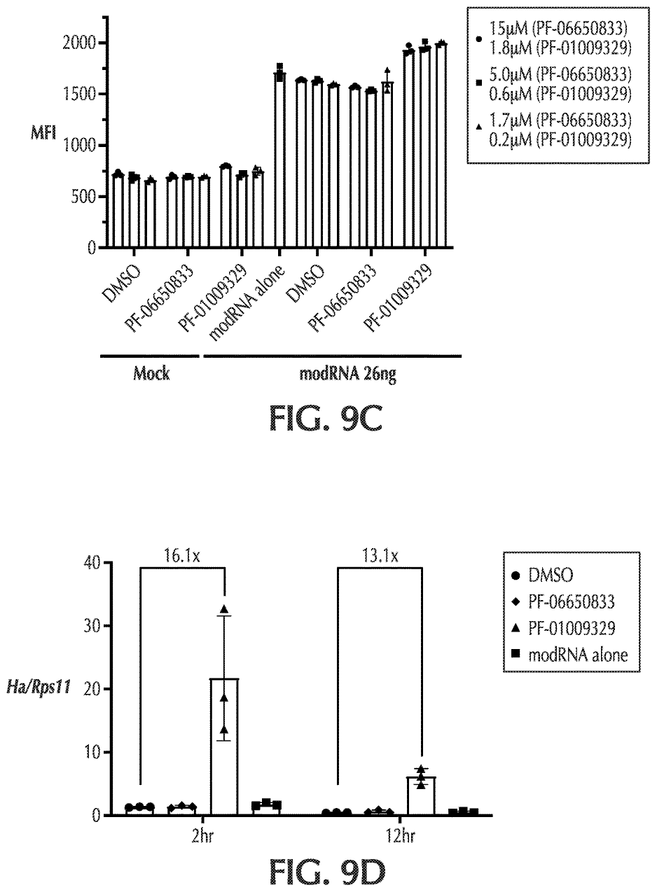

Relative transfection efficiency, as measured by the percentage of cells expressing HA, was lowest for saRNA alone-LNPs, without PF-01009329, peaking at a mean of 40.9% of live cells for the DMSO admixture control. Incorporation of admixed PF-01009329 at the 3× comparator concentration, significantly improved the percentage of antigen positive cells and EC50 values, as compared to the admixed DMSO control. Co-formulation of saRNA-LNPs with the hydrate form of PF-01009329 led to small molecule, dose dependent increases, in the percentage of cells expressing HA, and corresponding decreases in EC50 values. These data demonstrate that increasing the concentration of the small molecule in co-formulated LNPs, directly correlates to improved transfection efficiency of the current LNP formulations. Relative HA expression was quantitated via GMFI of HA immunostaining. All samples with PF-01009329 exhibited elevated GMFI values at LNP doses of 62 ng or higher. There was little difference between co-formulation and admixture conditions, or across small molecule doses, indicating that while the small molecule increases relative transfection efficiency, it likely does not participate in direct alterations of total protein expression at the level of translation. Cell viability correlated inversely with transfection efficiency, where a higher percentage of antigen expressing cells exhibited a lower overall viability (FIG. 8). Taken together, these data demonstrate that transfection efficiency of saRNA-LNPs increases concurrently with increases in PF-01009329 concentration. Furthermore, these data highlight that alternative forms of PF-01009329 can be used to load more of the small molecule into the current LNP formulations, and this should increase overall transfection efficiency, in vitro.

### 7. PF-01009329 Mode of Action

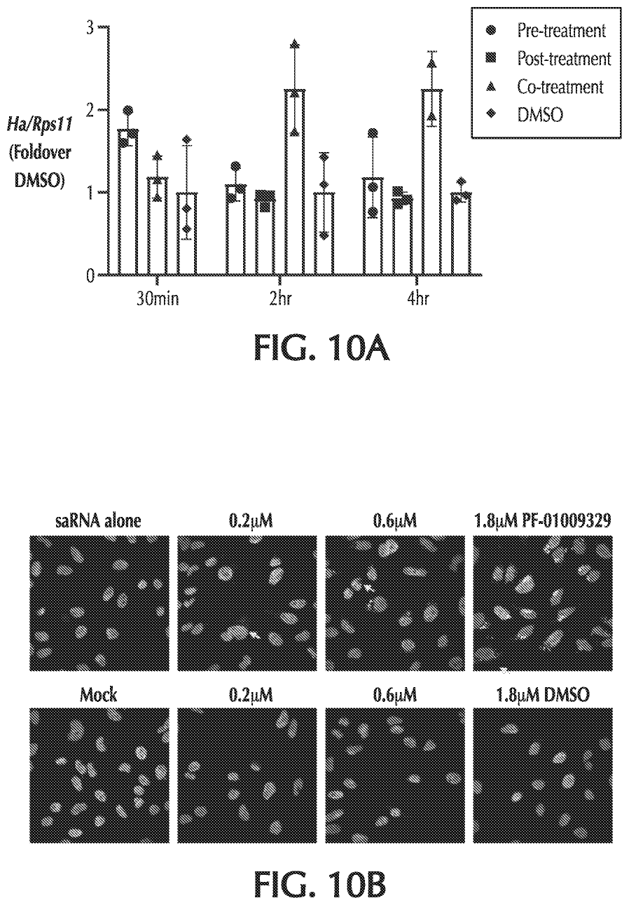

To better understand how the small molecule PF-01009329 was enhancing the presence of intracellular RNA, delivered via LNP treatment of cells (FIGS. 5D & 9D), a sequential administration experiment was designed. Cells were either pretreated with a vehicle control or PF-01009329, for one hour prior to modRNA-LNP addition, dosed simultaneously with an admixed modRNA-LNP+PF-01009329 sample, or exposed to modRNA-LNP treatment one hour prior to addition of PF-01009329 into the cell media. Total RNA was collected at 30 min (pre-treatment/co-treatment/DMSO only), 2 hours, and 4 hours post modRNA-LNP addition for all conditions and samples were analyzed via RT-qPCR. The presence of PF-01009329 in the media of cells, 1 hour prior to modRNA-LNP addition, led to a transient, nearly 2-fold increase in vaccine GOI transcripts, which was not observed at either 2 hours or 4 hours post LNP addition (3 hours and 5 hours post PF-01009329 addition). This indicates that PF-01009329 can enhance the presence of intracellular RNA delivered by LNPs even when the small molecule is added directly to the media of cells, rather than directly to LNP preparations. Treatment of cells with PF-01009329 following LNP addition (post-treatment), did not lead to increased intracellular vaccine transcripts, indicating that there is a spatial and/or temporal association between PF-01009329 and its ability to act either directly on cells or LNPs to enhance cellular RNA delivery. Interestingly, there was a delayed (relative to the pre-treatment condition) increase in intracellular vaccine transcripts when PF-01009329 was admixed with the LNPs, however, the boost in transcripts held at approximately 2-fold over the vehicle control alone, through the 4-hour timepoint (FIG. 10A). This indicates that enhancement of intracellular vaccine transcripts can be achieved through the use of PF-01009329, either via pre-treatment of cells, or direct incorporation into LNP, albeit with differential magnitudes and kinetics. Furthermore, these data indicate that the increases in intracellular vaccine transcripts, driven by addition of PF-01009329, are not specific to the saRNA platform, but are likely “nucleic acid cargo agnostic”.

To verify subcellular localization of saRNA after PF-01009329-mediated cellular uptake of saRNA-LNP, measurement of uptake kinetics by smFISH was performed. As shown in FIG. 10B, PF-01009329 induced higher cellular internalization of saRNA-LNP & dose-dependent cytoplasmic distribution of saRNA was detected at 4 hours post transfection. Together, this demonstrates that higher transfection efficiency of PF-01009329-containing LNPs associates with the improved saRNA potency.

1. A composition comprising at least one nucleic acid and at least one compound selected from any of the compounds shown in Table 1, or a pharmaceutically acceptable salt thereof.

2. The composition of claim 1 wherein the at least one nucleic acid is RNA.

3. The composition of claim 2, wherein the RNA is modRNA.

4. The composition of claim 2 wherein the RNA is saRNA.

5. The composition of claim 4, wherein the saRNA comprises at least one open reading frame that encodes a gene of interest.

6. The composition of claim 5, wherein the gene of interest is derived from influenza virus hemagglutinin (HA).

7. The composition of claim 6, wherein the saRNA further comprises a second reading frame that encodes a second gene of interest.

8. The composition of claim 7, wherein said second gene of interest is derived from influenza virus neuraminidase (NA).

9. The composition of claim 1, wherein said at least one compound modulates the signaling activity of RIG-1.

10. The composition of claim 1, wherein the at least one compound is selected from the group consisting of: zimlovisertib; 2,2′-(piperazine-1,4-diyl)bis(6,7-dimethoxyquinazolin-4-amine); 6,7-dimethoxy-2-{4-[6-(4-methylpiperazin-1-yl)pyrimidin-4-yl]piperazin-1-yl}quinazolin-4-amine; 2-[4-(4-amino-6,7-dimethoxyquinazolin-2-yl)piperazin-1-yl]-5-ethyl-1,3-thiazol-4(5H)-one; 6,7-dimethoxy-2-{4-[6-methyl-2-(pyrrolidin-1-yl)pyrimidin-4-yl]piperazin-1-yl}quinazolin-4-amine; and 2-{4-[4-(dimethylamino)-6-methylpyrimidin-2-yl]piperazin-1-yl}-6,7-dimethoxyquinazolin-4-amine, or a pharmaceutically acceptable salt thereof.

11. The composition of claim 10, wherein the at least one nucleic acid and the at least one compound are encapsulated in, bound to, or adsorbed on a cationic lipid.

12. The composition of claim 11, further comprising a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, a polycationic peptide, or a cationic nanoemulsion.

13. (canceled)

14. The composition of claim 1, wherein the at least one nucleic acid and the at least one compound are encapsulated in a lipid nanoparticle (LNP), wherein said LNP comprises at least one phospholipid.

15. (canceled)

16. (canceled)

17. (canceled)

18. The composition of claim 1, wherein the at least one nucleic acid and the at least one compound are encapsulated in a lipid nanoparticle (LNP), wherein said LNP comprises at least one cationic lipid.

19. The composition of claim 18, wherein the at least one cationic lipid is [(4-hydroxybutyl)azanediyl]di(hexane-6,1-diyl) bis(2-hexyldecanoate).

20. The composition of claim 1, wherein the at least one nucleic acid and the at least one compound are encapsulated in a lipid nanoparticle (LNP), wherein said LNP comprises at least one PEG lipid.

21. The composition of claim 20, wherein the at least one PEG lipid is 2-[(polyethylene glycol)-2000]-N,N-ditetradecylacetamide.

22. A method of increasing the expression of a protein encoded by a nucleic acid, comprising administering to a subject in need thereof the compositions according to claim 1.

23. The method of claim 22, wherein the compound is co-formulated with the nucleic acid.

24. The method of claim 22, wherein the compound is administered either before or after the nucleic acid is administered.
